SEQUENCE LISTING (2 sequences)

SEQ ID NO: 1 - protein, 213 aa, Artificial
  Humanized antibody
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, Artificial
  Humanized antibody
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2005/0089517 A1
(43) Pub. Date: Apr. 28, 2005

(54) Treatment of respiratory diseases with anti-IL-2 receptor antibodies

(76) Inventor: Richard S. Shames, Palo Alto, CA (US)

Correspondence Address: HOWREY SIMON ARNOLD & WHITE, LLP, c/o IP DOCKETING DEPARTMENT, 2941 FAIRVIEW PARK DRIVE, SUITE 200, FALLS CHURCH, VA, 22042-2924, US

(21) Appl. No.: 10/947,432

(22) Filed: Sep. 21, 2004

Related U.S. Application Data
(60) Provisional application No. 60/505,883, filed on Sep. 23, 2003.
(60) Provisional application No. 60/552,974, filed on Mar. 12, 2004.

(51) Int. Cl. A61K 39/395

(52) U.S. Cl. 424/141.1; 424/145.1

(57) ABSTRACT

The present invention provides a method of treating respiratory and allergic diseases. In particular, it provides a method for the treatment of asthma comprising administering to a subject a therapeutically effective amount of a pharmaceutical formulation comprising an antibody, wherein said antibody binds to IL-2 receptor.

### CROSS REFERENCE TO RELATED APPLICATIONS

The present application claims priority to U.S. provisional application 60/505,883 filed Sep. 23, 2003 and U.S. provisional application 60/552,974 filed Mar. 12, 2004, each of which is hereby incorporated by reference herein in its entirety.

### FIELD OF THE INVENTION

The present invention generally relates to the field of antibody therapeutics, particularly anti-IL-2 receptor antibodies, and to methods of treating T-cell mediated respiratory and allergic diseases, particularly, Th1- and Th2-cell mediated allergic diseases and/or symptoms, and most preferably asthma, with these antibody therapeutics.

### BACKGROUND OF THE INVENTION

T-cell activation and cytokine secretion play key roles in a range of respiratory and allergic diseases including, most notably, asthma. Asthma is a complex disorder characterized by airway inflammation associated with intermittent, reversible airway obstruction and airway hyper-responsiveness. Although its causes are unknown, airway inflammation involving lymphocytes, mast cells, eosinophils, and neutrophils are common features of all patients with chronic persistent asthma. Synthesis and release of cytokines, largely from activated T cells, initiate and sustain inflammatory processes in the airways (Drazen J. M. et al., J. Exp. Med. 183:1-5 (1996)). A variety of cytokines secreted by CD4⁺/CD25⁺ T cells are involved in chronic asthmatic inflammation, including IL-3, IL-4, IL-5, and granulocyte-macrophage colony-stimulating factor (Kon O. M. et al., Inflamm. Res. 48:516-23 (1999)). It also is likely that activated T cells are central to the initiation and regulation of airway repair processes in asthma that lead to airway fibrosis. Therefore, therapeutic strategies that are directed specifically at inhibiting activated T cells may be of benefit in asthmatic patients.

Autopsy studies, as well as data from bronchial biopsy specimens, confirm the presence of increased numbers of T cells in asthmatic airways. A postmortem study of 15 asthma patients revealed that the number of T cells in asthmatic airways was approximately twice that of 10 nonasthmatic, age-matched individuals (Azzawi M. et al., Am. Rev. Respir. Dis. 145:1477-82 (1992)). These cells were activated, as indicated by the expression of interleukin-2 (IL-2) receptors (CD25⁺), human leukocyte antigen-DR, very late antigen-1 and IL-5 mRNA expression. A study of the peripheral blood of severe asthma patients demonstrated the presence of increased numbers of CD4⁺/CD25⁺ T cells (Corrigan C. J. et al., Lancet 1:1129-32 (1988)). Studies of bronchoalveolar lavage from asthmatic patients also demonstrated increased numbers of activated CD25⁺ T cells, and increased levels of IL-2 and soluble IL-2 receptors (Alexander A. G. et al., J. Eur. Respir. 8:574-8 (1995); Park C. S. et al., Chest. 106:400-6 (1994); Walker C. et al., J. Allergy Clin. Immunol. 88:935-42 (1991)).

Daclizumab is an immunosuppressive, humanized immunoglobulin IgG1 monoclonal antibody produced by recombinant DNA technology. Daclizumab binds specifically to the alpha subunit (p55α, CD25, or Tac subunit) of the human high-affinity IL-2 receptor that is expressed on the surface of activated lymphocytes. The Tac subunit is expressed only after interaction with foreign antigen or with IL-2. Because daclizumab is made up of 90% human immunoglobulin sequences and only 10% murine sequences, its immunogenicity is low. The amino acid and nucleic acid sequences of daclizumab are disclosed in U.S. Pat. Nos. 5,530,101 and 5,693,761, each of which is hereby incorporated by reference herein in its entirety.

Daclizumab has been approved by the U.S. Food and Drug Administration for the prevention of renal allograft rejection in patients receiving concomitant immunosuppression with cyclosporine and steroids, with or without azathioprine or mycophenolate mofetil (ZENEPAX®, Package Insert,Roche Laboratories (2000)). The incidence of acute rejection at 6 months posttransplant was reduced by up to 40% in patients who received 5 doses (1 mg/kg) of daclizumab as compared to placebo in 2 double-blind, controlled trials of patients who were receiving their first cadaver renal allograft. There was no additional toxicity associated with the use of daclizumab, nor was there any increase in opportunistic infections or lymphomas (Vincenti F. et al., J. Med. N. Engl. 338:161-5 (1998); Nashan B. et al., Transplantation 67:110-5 (1999)).

Daclizumab also has been evaluated in patients with autoimmune uveitis who were receiving concomitant immunosuppression with cyclosporine and/or steroids. Patients were weaned off their systemic immunosuppressive agents, while ultimately receiving daclizumab infusions every 4 weeks. Daclizumab appeared to prevent the expression of severe sight-threatening intraocular inflammatory disease in 8 of 10 patients treated over a 12-month period, with no deterioration in visual acuity. The therapy was well tolerated (Nussenblatt R. B. et al., Proc. Nat'l. Acad Sci U.S.A 96:7462-6 (1999)).

A Phase I, multiple-dose study of daclizumab in 19 patients with moderate to severe psoriasis showed that the drug was well tolerated, with no specific adverse events associated with its administration. Patients were infused with daclizumab (2 mg/kg loading dose, followed by 1 mg/kg) at weeks 2, 4, 8, 12, and 16. This study showed a consistent blockade of CD25 in peripheral blood and tissue during the first 4 weeks of therapy while the dosing was every 2 weeks. Patients with a pretreatment PASI score of <36 showed a mean reduction in severity by 30% at 8 weeks (P=0.02). Variable desaturation of receptors began after 4 weeks, which correlated with a reversal in disease improvement. During the 16 weeks of treatment, there was a 44.8% decrease in expression of the IL-2 receptor α-subunit. The absolute T-cell counts showed no significant changes during the course of the study. No significant adverse events were produced by daclizumab during this study (Krueger J. G., et al., J. Am. Acad. Dermatol. 43:448-58 (2000)).

In view of the prevalence of respiratory diseases, particularly T-cell mediated diseases such as asthma, and the lack of effective methods for treating respiratory diseases, it is a highly desirable goal of this invention to provide more effective therapeutic methods and agents. New treatment methods and agents are especially needed for the more severe, refractory types of asthma, and other T-cell mediated respiratory and allergic diseases, that do not respond to conventional nonspecific immunosuppression therapy. The present invention encompasses methods for the treatment of T-cell mediated respiratory and allergic diseases, particularly respiratory diseases such as asthma, but also including a range of Th1- and Th2-cell mediated allergic diseases and/or symptoms. The method involves administering anti-IL-2 receptor antibodies, and preferably the humanized antibody, daclizumab, and antibodies that bind the same IL-2 receptor epitope as daclizumab. As demonstrated by the results of the Phase II clinical study disclosed herein, daclizumab offers superior clinical efficacy and long-lasting beneficial results for treatment of moderate to severe asthma compared to the existing treatment approaches.

### SUMMARY OF THE INVENTION

The present invention provides methods for the therapeutic or prophylactic treatment of a T-cell mediated disease, particularly a respiratory and/or allergic disease caused or exacerbated by IL-2 receptor-mediated activation, such as a Th1- or Th2-cell mediated allergic disease or symptom. The methods for the therapeutic or prophylactic treatment of a respiratory and/or allergic disease and/or symptoms comprise administering to a patient in need of such treatment a therapeutically or prophylactically effective amount of a pharmaceutical formulation comprising an antibody that binds specifically to an IL-2 receptor. In another embodiment, the method of treatment further comprises administering to the patient a concomitant medication for the targeted disease.

In one preferred embodiment, the method of the invention may be applied wherein the disease is selected from the group consisting of asthma, allergic rhinitis, atopic dermatitis, nasal polyposis, Churg-Strauss syndrome, sinusitis, and chronic obstructive pulmonary disease (COPD).

In other preferred embodiments, the treatment method of the invention may be applied wherein the disease is a Th2-cell mediated allergic disease and/or symptom selected from the group consisting of asthma, atopic dermatitis, anaphylaxis, urticaria (hives), allergic rhinitis, nasal polyposis, sinusitis, allergic conjunctivitis, skin allergy, eczema, hay fever, allergic gastroenteritis, or Churg-Strauss syndrome.

In another embodiment, the treatment method of the invention may be applied wherein the disease is a Th1-cell mediated disease and/or symptom selected from the group consisting: interstitial lung diseases (ILD) (e.g., idiopathic pulmonary fibrosis), hypersensitivity lung diseases, and hypersensitivity pneumonitis

In a preferred embodiment, the method of treatment is carried out on a patient with mild, moderate, or severe asthma of any type, etiology or pathogenesis. In a particularly preferred embodiment, the method is carried out on a patient with chronic, persistent asthma, or patients with moderate to severe asthma. In particular, the method may be used for for those patients whose asthma is suboptimally controlled by corticosteroids. In other specific embodiments, the methods of the present invention may be carried out to treat patients with an atopic or non-atopic asthma including but not limited to: allergic asthma, bronchitic asthma, exercise-induced asthma, occupational asthma, asthma induced following bacterial infection, and “wheezy-infant syndrome.”

In one embodiment, the method of treating asthma further comprises administering to the patient a concomitant asthma medication. In preferred embodiments, the concomitant asthma medication may be selected from group consisting of inhaled or oral steroids, leukotriene modifying agents, inhaled or oral β2-agonists, and inhaled ipratroprium. In one preferred embodiment, the concomitant asthma medication is an inhaled steroid selected from the group consisting of beclomethasone, budesonide, flunisolide, fluticasone, triamcinolone, mometasone and acetonide.

In preferred embodiments, the methods of the present invention are carried out using a monoclonal antibody, and in particular, a chimeric, humanized or human antibody. In some embodiments of the invention, the antibody neutralizes one or more of the biological activities of the IL-2 receptor.

In particularly preferred embodiments, the methods of treatment of the present invention are carried out wherein the antibody that specifically binds IL-2 receptor is daclizumab, or an antibody that binds to the same epitope as daclizumab. In another embodiment, the methods of treatment may be carried out using an antibody comprising CDRs at least 60% identical in amino acid sequence to those of daclizumab. In other embodiments, the methods may be carried out wherein the CDRs of the anti-IL2 receptor antibody is at least 70%, 75%, 80%, 85%, 90%, 91%, 92%, 93%, 94%, 95%, 96%, 97%, 98%, or even 99% identical in amino acid sequence to the CDRs of daclizumab. In preferred embodiments, the methods of the present invention are carried out wherein the antibody has a binding affinity for said human IL-2 receptor of at least 10⁸ M⁻¹, and more preferably, at least 10⁹ M⁻¹.

In preferred embodiments, the methods of the present invention are carried out wherein the pharmaceutical formulation, comprising an anti-IL2 receptor antibody, is administered parenterally, intravenously, intramuscularly, or subcutaneously. In a preferred embodiment, the formulation comprises daclizumab. In a preferred embodiment, the method is carried out wherein the pharmaceutical formulation is a liquid comprising about 100 mg/ml daclizumab, about 20-60 mM succinate buffer (or 20-70 mM histidine buffer), having pH from about 5.5 to about 6.5, about 0.01% -0.1% polysorbate, and a tonicity buffer that contributes to isotonicity (e.g. about 75-150 mM NaCl, or about 1-100 mM MgCl₂).

In other embodiments, the methods of the present invention are carried out wherein the therapeutically effective amount of the pharmaceutical formulation is between about 0.001 mg/kg to 10 mg/kg, and preferably between about 0.5 mg/kg to 4.0 mg/kg. In some embodiments, the therapeutically effective amount is a fixed dose of between about 100 mg and 200 mg.

### BRIEF DESCRIPTION OF THE DRAWINGS

FIG. 1 depicts the study schema for the Phase II study of daclizumab in patients with moderate to severe, chronic, persistent asthma described in Example 1.

FIG. 2 depicts a schematic for the inhaled corticosteroid titration during the Run-in phase of the Phase II study of daclizumab in patients with moderate to severe, chronic, persistent asthma described in Example 1.

FIG. 3 depicts a table listing the schedule of patient assessments for the Phase II study described in Example 1.

### DETAILED DESCRIPTION OF THE INVENTION

Definitions

As used herein, “antibody” refers to an immunoglobulin molecule that specifically binds to, or is immunologically reactive with a particular antigen, and includes both polyclonal and monoclonal antibodies. The term also includes genetically engineered or otherwise modified forms of immunoglobulins, such as chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)₂, Fab, Fv, rIgG, and scFv fragments. The term “scFv” refers to a single chain Fv antibody in which the variable domains of the heavy chain and of the light chain of a traditional two chain antibody have been joined to form one chain. Typically, a linker peptide is inserted between the two chains to allow for proper folding and creation of an active binding site. In addition, the term “antibody,” as used herein, is also intended to encompass mixtures of more than one antibody reactive with a specific antigen (e.g., a cocktail of different types of monoclonal antibodies reactive with IL-2 receptor).

The terms “specific binding,” “selective binding,” “specifically reactive,” or “specifically immunoreactive,” as used herein, refer to a binding reaction that may be used to determine the presence of the antibody in a heterogeneous population of proteins and other biological molecules. In other words, it is a binding reaction where the antibody does not cross react substantially with any antigen other than the one specified. Thus, for example, specific binding occurs where the antibody binds to the desired antigen with an affinity at least two times greater than background (i.e. nonspecific/cross-reacting binding level) and more typically more than 10 to 100 times greater than background. Specific binding of an antibody to a desired antigen generally requires an antibody that has been selected for that particular antigen. For example, polyclonal antibodies raised to specifically bind to a particular protein, or its polymorphic variants, alleles, orthologs, conservatively modified variants, splice variants, can be selected to obtain only those antibodies that are specifically immunoreactive with the selected protein (e.g. IL-2 receptor) and not with other proteins. This selection may be achieved by subtracting out antibodies that cross-react with other molecules. A variety of immunoassay formats may be used to select antibodies specifically reactive with a particular protein. For example, solid-phase ELISA immunoassays are routinely used to select antibodies specifically reactive with a protein (see, e.g., Harlow & Lane, Antibodies, A Laboratory Manual (1988) for a description of immunoassay formats and conditions that can be used to determine specific immunoreactivity).

“Antibodies of IgG class” as used herein refers to antibodies of IgG1, IgG2, IgG3, and IgG4. The numbering of the amino acid residues in the heavy and light chains is that of the EU index (Kabat, et al., “Sequences of Proteins of Immunological Interest”, 5^{th} ed., National Institutes of Health, Bethesda, Md. (1991); the EU numbering scheme is used herein).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 6-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Two antibodies are said to bind to the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody.

As used herein, “V_{H}” or a “VH” refer to the variable region of an immunoglobulin heavy chain of an antibody, including the heavy chain of an antigen binding fragment of an antibody, e.g., Fv, scFv, or Fab. References to “V_{L}” or a “VL” refer to the variable region of an immunoglobulin light chain, including the light chain of an antigen binding fragment of an antibody e.g., Fv, scFv , dsFv or Fab.

Antibody light and heavy chain variable regions contain four “framework” regions interrupted by three hypervariable regions, also called “complementarity-determining regions” or “CDRs.” The extent of the framework regions and CDRs are well-known to those of ordinary skill in the art (see e.g. Kabat, et al., “Sequences of Proteins of Immunological Interest”, 5^{th} ed., National Institutes of Health, Bethesda, Md. (1991)). The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The term “monoclonal antibody” as used herein is not limited to antibodies produced through hybridoma technology but refers to an antibodies derived from a single clone, including any eukaryotic, prokaryotic, or phage clone, and not the method by which it is produced. Monoclonal antibodies useful with the present invention may be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563-681 (both of which are incorporated herein by reference in their entireties). Production of antibodies by selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

The term “genetically altered antibodies” refers to antibodies wherein the amino acid sequence has been varied from that of a parent (i.e. unaltered) antibody. Thus, the amino acid sequences of the anti-IL2 receptor antibodies useful with the methods of the present invention are not confined to the sequences found in natural antibodies; antibodies can be redesigned to obtain desired characteristics using well-known recombinant DNA techniques. The possible variations range from the changing of just one or a few amino acids to the complete redesign of, for example, the variable or constant region. Changes, by site-directed mutation, in the constant region may be made in order to improve or alter the functional characteristics of a therapeutic antibody such as immunogenicity, pharmacokinetic characteristics (e.g. serum half-life), complement fixation, interaction with membranes and other effector functions. Generally, changes to the antibody variable region may be made in order to improve the antigen binding characteristics.

A “substantially identical constant region” refers to an antibody constant region wherein at least about 85-90%, and preferably at least 95% of the amino acid sequence is identical to a natural or unaltered antibody constant region.

The term “chimeric antibody,” as used herein, refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Methods for producing chimeric antibodies are well-known to those of ordinary skill in the art. See e.g., Morrison et al., Science 229:1202-1207 (1985); Oi et al., BioTechniques 4:214-221 (1986); Gillies et al., J. Immunol. Methods 125:191-202 (1989); and U.S. Pat. Nos. 5,807,715; 4,816,567; and 4,816,397, each of which is hereby incorporated herein by reference in its entirety.

The term “humanized antibody” refers to an immunoglobulin comprising a human framework, at least one and preferably all CDRs from a non-human antibody, and in which any constant region present is substantially identical to a human immunoglobulin constant region, i.e., at least about 85-90%, and preferably at least 95% identical. Hence, all parts of a humanized immunoglobulin, except possibly the CDRs, are substantially identical to corresponding parts of one or more native human immunoglobulin sequences. Accordingly, such humanized antibodies are chimeric antibodies, wherein substantially less than an intact human variable domain has been substituted by the corresponding sequence from a non-human species. Framework residues in the human framework regions may be substituted with the corresponding residue from the CDR donor antibody to alter, preferably improve, antigen binding. These framework substitutions may be identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos: 5,530,101; 5,585,089; 5,693,761; 5,693,762; 6,180,370 (each of which is incorporated by reference in its entirety). Antibodies may be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489-498 (1991); Studnicka et al., Prot. Eng. 7:805-814 (1994); Roguska et al., Proc. Natl. Acad. Sci. 91:969-973 (1994), and chain shuffling (U.S. Pat. No. 5,565,332), all of which are hereby incorporated by reference in their entireties.

The term “human antibodies” refers to an antibodies comprising both a human variable and constant region. Human antibodies may be desirable for therapeutic treatment of human patients according to the methods of the present invention. Human antibodies can be made or obtained by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix, Inc. (Fremont, Calif.) and Medarex (Princeton, N.J.) can be engaged to provide human antibodies directed against a selected antigen using technology similar to that described above. Completely human antibodies that recognize a selected epitope also can be generated using a technique referred to as “guided selection.” In this approach a selected non-human monoclonal antibody, e.g., a mouse antibody, is used to guide the selection of a completely human antibody recognizing the same epitope (Jespers et al., Biotechnology 12:899-903 (1988).

The term “primatized antibody” refers to an antibody comprising monkey variable regions and human constant regions. Methods for producing primatized antibodies are known in the art. See e.g., U.S. Pat. Nos. 5,658,570; 5,681,722; and 5,693,780, which are incorporated herein by reference in their entireties.

The term “amino acid” refers to naturally occurring and synthetic amino acids, as well as amino acid analogs and amino acid mimetics that function similarly to the naturally occurring amino acids. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Naturally occurring amino acids” refers to those encoded by the genetic code, as well as those amino acids that are later modified, e.g., hydroxyproline, γ-carboxyglutamate, and O-phosphoserine.

“Amino acid analogs” refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or a modified amide group, but retain the same basic chemical structure as a naturally occurring amino acid. “Amino acid mimetics” refers to chemical compounds that have a structure that is different from the general chemical structure of an amino acid, but that functions similarly to a naturally occurring amino acid.

The terms “polypeptide,” “peptide” and “protein” are used interchangeably herein to refer to a polymer of amino acid residues linked by peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The terms “identical” or percent “identity,” in the context of two or more amino acid or nucleotide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., description of BLAST at NCBI web site located at www.ncbi.nlm.nih.gov). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. The well-known algorithms for measuring sequence identity can account for gaps and the like. Preferably, identity exists over a region that is at least about 25 amino acids or nucleotides in length, or more preferably over a region that is 50-100 amino acids or nucleotides in length.

“Conservatively modified variants” as used herein, may apply to variants in amino acid or nucleic acid sequences. With respect to amino acid sequences, a conservatively modified variant sequences includes sequences with substitutions, deletions or additions that add or delete one, or a small percentage of, amino acids, or substitute one or a small percentage of amino acids with a chemically similar amino acid. Conservative substitution tables providing functionally similar amino acids are well known in the art. Typical conservative substitutions of one amino acid for another include the following: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Thomas E. Creighton, “Proteins: Structures and Molecular Properties,” (ISBN 071677030X, W. H. Freeman, 1992)). Such conservatively modified variants of amino acid sequences are in addition to and do not exclude polymorphic variants, interspecies homologs, and alleles of the invention.

With respect to nucleic acid sequences, conservatively modified variants refers to sequences that encode identical or essentially identical amino acid sequences (e.g. nucleic acid sequences that encode conservatively modified variant amino acid sequences. Where the nucleic acid sequence does not encode an amino acid sequence, to essentially identical or associated, e.g., naturally contiguous, sequences. Because of the degeneracy of the genetic code, there are a large number of conservatively modified variant nucleic acid sequences encoding most proteins. For instance, the codons GCA, GCC, GCG, and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein, which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

“Carriers” as used herein include pharmaceutically acceptable carriers, excipients, or stabilizers, which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids, antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrans; chelating agents such as EDTA; sugar alcohols such as mannitol or sorbitol; salt-forming, counter-ions such as sodium; and/or nonionic surfactants such as TWEEN™, polyethylene glycol (PEG), and PLURONICS™.

As used herein, “therapeutically effective amount” refers to the amount of a drug, pharmacologically active agent, pharmaceutical formulation or composition that is sufficient to cure, alleviate, attenuate or at least partially arrest a disease and/or its symptoms, and/or complications.

“Treatment” refers to both therapeutic treatment and prophylactic or preventative measures. Those in need of treatment include those already with the disorder as well as those in which the disorder is to be prevented.

A “subject,” or “patient” is used interchangeably herein, which refers to a vertebrate, preferably a mammal, more preferably a human.

The term “derived from,” as used herein, means “obtained from” or “produced by” or “descended from.”

Description of the Invention

Disease Indications

The present invention provides methods for treating or preventing a respiratory disease in a subject in need of such a treatment or prevention. The therapeutic method comprises administering a therapeutically effective amount of an antibody capable of specifically inhibiting the binding of IL-2 to the IL-2 receptor, and/or inhibiting IL-2-mediated activation of lymphocytes. In one preferred embodiment, the targeted respiratory disease is asthma. The present method may be used for the treatment of mild, moderate, or severe asthma of any type, etiology or pathogenesis. As demonstrated by the data disclosed herein, the method is particularly effective against chronic, persistent asthma, particularly in those patients whose asthma is suboptimally controlled by corticosteroids. Furthermore, the methods of the present invention may be employed for the treatment of either atopic or non-atopic asthma, including allergic asthma, bronchitic asthma, exercise-induced asthma, occupational asthma, asthma induced following bacterial infection, “wheezy-infant syndrome” (i.e. wheezing symptoms observed particularly at night in subjects of less than 4 or 5 years of age who may also be identified as incipient or early-phase asthmatics), and other non-allergic asthmas.

The efficacy of a treatment for asthma may be measured by methods well-known in the art. The method of asthma treatment of the present invention has been found to yield one or more of the following results indicating efficacy: increase in pulmonary function (spirometry), decrease in asthma exacerbations, increase in morning peak expiratory flow rate, decrease in rescue medication use, decrease in daytime and nighttime asthma symptoms, increase in asthma-free days, increase in time to asthma exacerbation, and increase in forced expiratory volume in one second (FEV₁).

The method of treatment may further comprise administering a concomitant asthma medication (e.g. an inhaled steroid) to the patient. Preferably, the steroid is one used in the treatment of a respiratory disease, such as asthma. More preferably, the steroid is one or more selected from the group consisting of beclomethasone, budesonide, flunisolide, fluticasone, and triamcinolone. In one embodiment, the steroid can be in the same formulation as the anti-IL-2 receptor antibody. In another embodiment, the steroid is administered to the patient separate from the administration of the anti-IL-2 receptor antibody. In one embodiment, the steroid is administered in an amount that is not therapeutically sufficient to treat or prevent the respiratory disease, such as asthma, when administered in the absence of the anti-IL-2 receptor antibody.

In one embodiment, the steroid is administered in an amount that is not sufficient to cause any adverse effects or flares in the patient. Preferably, the amount of steroid administered is the highest dosage possible that is not sufficient to cause any adverse effects or flares in the patient.

Based on the demonstrated efficacy of the anti-IL2 receptor antibody, daclizumab to reduce eosinophil levels in severe asthma patients, the methods of the present invention may reasonably be expected to be useful for the treatment of other respiratory or allergic diseases and/or symptoms. Increased eosinophil levels are a hallmark of many T-cell mediated allergic diseases. Daclizumab also is known to reduce production of T-cell associated cytokines. Thus, those diseases or symptoms associated with the T-cell mediated inflammatory responses may be treated with daclizumab (or other anti-IL2 receptor antibodies) according to the methods of the present invention.

T-cell mediated respiratory and/or allergic diseases and/or symptoms that may be treated include both Th1-cell and Th2-cell mediated diseases. For example, specific Th2-cell mediated allergic diseases and/or symptoms that may be treated with daclizumab according to the method of the present invention include, but are not limited to: asthma, atopic dermatitis, anaphylaxis, urticaria (hives), allergic rhinitis, nasal polyposis, sinusitis, allergic conjunctivitis, skin allergy, eczema, hay fever, allergic gastroenteritis, Churg-Strauss syndrome. Th1-cell mediated respiratory diseases and/or symptoms that may be treated with daclizumab treatment method of the present invention include, but are not limited to: interstitial lung diseases (ILD) (e.g., idiopathic pulmonary fibrosis), hypersensitivity lung diseases, and hypersensitivity pneumonitis.

In addition, interstitial lung diseases (ILD) often are associated with a wide range of systemic autoimmune diseases including, but not limited to: rheumatoid arthritis, systemic lupus erythematosus, ankylosing spondylitis, systemic sclerosis, Sjogren's syndrome, pollinosis, scleroderma, sarcoidosis, polymyositis or dermatomyositis. Consequently, the anti-IL2 receptor antibody method of treatment of the present invention may be useful in the treatment of the disease and/or symptoms associated with these systemic autoimmune diseases, alone or in combination with other treatments.

There is a range of eosinophil-mediated diseases and/or symptoms including, but not limited to: pulmonary eosinophilia, eosinophilic-myalgia syndrome, tropical eosinophilia, hypereosinophilic syndrome, and parasitic infections, including, but not limited to schistosomiasis. Many of these eosinophil-mediated diseases currently are being treated with IL-5 based therapeutics. Based on its efficacy in attenuating eosinophil levels in T-cell mediated diseases (e.g. asthma), the anti-IL2 receptor antibody method of treatment of the present invention may also be useful in treating these eosinophil-mediated diseases alone, or in combination with other treatments.

Chronic obstructive pulmonary (or airways) disease (COPD) is a condition defined physiologically as airflow obstruction that generally results from a mixture of emphysema and peripheral airway obstruction due to chronic bronchitis. COPD is the fifth leading cause of death in the world and the need for effective drugs and treatment methods is extremely high. COPD is a subgroup of the chronic lung diseases which also includes asthma and which are characterized by a chronic inflammation and/or fibrosis of the airway tissue. Many pathophysiological features are shared among these diseases, thus, the anti-IL2 receptor antibody method of treatment of the present invention may reasonably be expected to be useful for the treatment of COPD.

Anti-IL-2 Receptor Antibodies

Anti-IL-2 receptor antibodies for use in the present invention include antibodies that bind to any epitope of the IL-2 receptor. Preferably, the epitope is found on the alpha subunit (p55 alpha, CD25, or Tac subunit) of the IL-2 receptor. They include natural anti-IL-2 receptor antibodies (the antibodies that are produced by a host animal) and recombinant anti-IL-2 receptor antibodies. The anti-IL-2 receptor antibodies of all species origins are included. Non-limiting exemplary natural anti-IL-2 receptor antibodies include anti-IL-2 receptor antibodies derived from human, chicken, goats, and rodents (e.g., rats, mice, hamsters and rabbits), including transgenic rodents genetically engineered to produce human antibodies (see, e.g., U.S. Pat. No. 6,300,129 B1 (Lonberg et al.), and U.S. Pat. No. 6,114,598 (Kucherlapati, et al.), each of which is hereby incorporated by reference herein in its entirety). Antibodies useful in the present invention also may be made using phage display methods (see, e.g., U.S. Pat. No. 5,427,908 (Dower et al.) and U.S. Pat. No. 5,969,108 (Bonnert et al.), each of which is hereby incorporated by reference herein in its entirety). For use in human patients, the antibodies must bind specifically to human IL-2 receptor. The antibodies should have binding affinity for IL-2 receptor of at least 10⁷ M⁻¹ but preferably at least 10⁸ M⁻¹, more preferably at least 10⁸ M⁻¹, most preferably 10⁹ M⁻¹ and ideally 10¹⁰ M⁻¹ or higher. The affinity of the antibodies may be increased by in vitro mutagenesis using phage display or other methods (see, e.g., Co, et al., U.S. Pat. No. 5,714,350, which is hereby incorporated by reference herein in its entirety).

Preferably, the antibody binds specifically to the alpha subunit (p55 alpha, CD25, or Tac subunit) of an IL-2 receptor. More preferably, the IL-2 receptor is an IL-2 receptor that is expressed on the surface of an activated lymphocyte. Preferably, the lymphocyte is a T-cell.

Preferably, the antibodies will neutralize at least one but most preferably all biological properties of IL-2 receptor, for example, IL-2 mediated activation of lymphocytes. The antibodies will generally inhibit or block binding of IL-2 receptor to IL-2. The antibodies should inhibit proliferation and activation of the activated T-cells, or induce apoptosis of the activated T-cells.

Preferably, the antibodies do not specifically bind Fcγ receptors and thereby the antibodies do not substantially activate mitogenic responses in T-cells in most or all patients. Preferably, the antibodies have the following desirable properties as immunosuppressive agents: they can suppress immune responses of T-cells without inducing mitogenic activity resulting in harmful release of cytokines, at least in most (e.g. at least 67%, 75%, 90% or 95% ) patients.

The polyclonal forms of anti-IL-2 receptor antibodies may be produced in non-human host animals by immunization with human IL-2 receptor. The monoclonal antibodies can be produced by immunization and hybridoma methodologies well known in the art (see e.g., Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563-681). For example, production and initial screening of monoclonal antibodies to yield those specific for the IL-2 receptor can be carried out as described in Uchiyama et al., J. Immunol. 126 (4), 1393 (1981). Another suitable monoclonal antibody is the M7/20 monoclonal antibody described by Gaulton et al. (Clin. Immunol. and Immunopath (1985)) which is a monoclonal rat anti-mouse κ, u, Ig antibody specific for the IL-2 receptor (see also, U.S. Pat. No. 5,916,559, which is hereby incorporated by reference herein in its entirety). Another suitable monoclonal antibody is the 2A3 monoclonal antibody produced by hybridoma ATCC HB-8555, which is disclosed in U.S. Pat. No. 4,845,198, which is hereby incorporated by reference herein in its entirety. Other suitable anti-IL-2 antibodies are described in U.S. Pat. Nos. 4,411,993 and 4,473,493, each of which is hereby incorporated by reference herein in its entirety.

Recombinant DNA techniques also may be used to produce recombinant anti-IL-2 receptor antibodies useful with the present invention. The variable and/or constant region amino acid sequences of such recombinant antibodies need not be genetically altered but may be identical to the sequences found in a natural antibody. Recombinant anti-IL-2 receptor antibodies useful with the present invention include antibodies produced by any expression system including both prokaryotic and eukaryotic expression systems. Exemplary prokaryotic systems are bacterial systems that are typically capable of expressing exogenously introduced nucleic acid sequences. Illustrative eukaryotic expression systems include fungal expression systems, viral expression systems involving eukaryotic cells such as insect cells, plant-cells and especially mammalian cells (such as CHO cells and myeloma cells such as NS0 and SP2/0) which are well-known to those of ordinary skill in the art. See e.g., Morrison et al., Science 229:1202-1207 (1985); Oi et al., BioTechniques 4:214-221 (1986); Gillies et al., J. Immunol. Methods 125:191-202 (1989); and U.S. Pat. Nos. 5,807,715; 4,816,567; and 4,816,397, each of which is hereby incorporated herein by reference, in its entirety. The antibodies may also be produced by chemical synthesis. However they are produced, the anti-IL-2 receptor antibodies may be purified by methods well-known in the art, such as filtration, chromatography (e.g., affinity chromatography such as by protein A, cation exchange chromatography, anion exchange chromatography, and gel filtration). Typically, the minimum acceptable purity of the antibody for use in pharmaceutical formulations will be 90%, with 95% preferred, 98% more preferred and 99% or higher most preferred.

Alternatively, the variable and/or constant region sequences of the recombinant construct may be genetically altered. Preferably, the genetically altered anti-IL-2 receptor antibodies used in the present invention include chimeric or humanized antibodies that bind to and neutralize IL-2 receptor. An exemplary, preferred humanized anti-IL-2 receptor antibody is daclizumab. The amino acid and nucleotide sequences of daclizumab are disclosed in U.S. Pat. Nos. 5,530,101 and 5,693,761, each of which is hereby incorporated by reference herein in its entirety. The amino acid sequences of the daclizumab mature light and heavy chains are shown below:

(SEQ ID NO:1)

Daclizumab mature kappa light chain
DIQMTQSPSTLSASVGDRVTITCSASSSISYMHWYQQKPGKAPKLLIYTT
SNLASGVPARFSGSGSGTEFTLTISSLQPDDFATYYCHQRSTYPLTFGQG
TKVEVKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

(SEQ ID NO:2)

Mature gamma-1 heavy chain
QVQLVQSGAEVKKPGSSVKVSCKASGYTFTSYRMHWVRQAPGQGLEWIGY
INPSTGYTEYNQKFKDKATITADESTNTAYMELSSLRSEDTAVYYCARGG
GVFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFELYSKLTVDKSRWQQGNVFSCSVMHEALHNHYTQKSLSLSPGK

Daclizumab (commercially available as ZENAPAX®) is a humanized monoclonal antibody that binds specifically to the alpha subunit (p55 alpha, CD25, or Tac subunit) of the human high-affinity IL-2 receptor that is expressed on the surface of activated lymphocytes. ZENAPAX® was created by Protein Design Labs, Inc. (hereafter “PDL”; Fremont, Calif.) and developed and marketed by Roche Laboratories (Hoffmann-La Roche Inc., Nutley, N.J.). Daclizumab in its current clinical embodiment is an IgG1 isotype antibody, however, an IgG2M3 isotype version of daclizumab may also be produced that exhibits similar therapeutic characteristics.

Other preferred antibodies include those that bind to the same epitope of the IL-2 receptor as daclizumab. Preferably, the antibody that binds to the same epitope of the IL-2 receptor as daclizumab has an amino acid sequence at least 60% identical to the amino acid sequence of daclizumab. In other preferred embodiments, the amino acid sequence is at least 70%, 75%, 80%, 85%, 90%, 95%, 98%, 99% or 100% identical to the sequence of daclizumab.

In other preferred embodiments, the antibody that binds to the same epitope of the IL-2 receptor as daclizumab has a CDR with an amino acid sequence at least 60% identical to the amino acid sequence of the CDR of daclizumab. In other preferred embodiments, the CDR amino acid sequence is at least 70%, 75%, 80%, 85%, 90%, 95%, 98%, 99% or 100% identical to the CDR sequence of daclizumab. The anti-IL-2 receptor antibodies may be of any of the recognized isotypes, but the four IgG isotypes are preferred, with IgG2 especially preferred.

The methods of the present invention also may be carried out using genetically altered anti-IL-2 receptor antibodies, including chimeric antibodies that bind to and neutralize IL-2 receptor, or prevent the IL-2 receptor from binding IL-2. Preferably, the chimeric antibodies comprise a variable region derived from a mouse or rat and a constant region derived from a human so that the chimeric antibody has a longer half-life and is less immunogenic when administered to a human subject. The method of making chimeric antibodies is known in the art.

The present invention also includes the use of fragments of anti-IL-2 receptor antibodies that retain the binding specificity of the complete anti-IL-2 receptor antibodies described supra. Examples include, but are not limited to, the heavy chains, the light chains, and the variable regions as well as Fab and (Fab′)₂ of the antibodies described herein.

The methods of the present invention also may be carried out using anti-IL2 receptor antibodies that are modified (e.g. by site-directed mutagenesis) but functionally equivalent (e.g. exhibit comparable IL-2 receptor binding affinity) to the above-described antibodies. For example, the antibodies may be modified to have improved stability (e.g. serum half-life) and/or therapeutic efficacy. Examples of modified antibodies include those with conservative substitutions of amino acid residues, and one or more deletions or additions of amino acids, which do not significantly deleteriously alter the antigen binding utility. Substitutions can range from changing or modifying one or more amino acid residues to complete redesign of a region as long as the therapeutic utility is maintained (e.g. specific binding capacity). In one preferred embodiment, daclizumab (or any other anti-IL2 receptor binding antibody) may be generated with site directed mutations in the FcRn binding region that extend significantly serum half-life, as described in U.S. patent application Ser. No. 10/687,118, filed Oct. 15, 2003, which is hereby incorporated by reference herein. Antibodies of this invention may also be modified post-translationally (e.g., acetylation, and phosphorylation) or synthetically (e.g., the attachment of a labeling group). Fragments of these modified antibodies that retain the binding specificity can also be used.

Pharmaceutical Formulations or Compositions

The antibodies of the invention may be formulated in pharmaceutical compositions. Thus, the present invention also provides methods and compositions for administering a therapeutically effective dose of an anti-IL2 receptor antibody. The exact dose will depend on the purpose of the treatment, and will be ascertainable by one of ordinary skill in the art using well-known techniques (see e.g., Ansel et al., “Pharmaceutical Dosage Forms and Drug Delivery,” (6^{th} Ed., Media, Pa.: Williams & Wilkins, 1995); “Pharmaceutical Dosage Forms” (Vols. 1-3, ISBN nos. 0824785762, 082476918X, 0824712692, 0824716981) eds. Lieberman et al. (New York: Marcel Dekker, Inc., 1992); Loyd V. Allen, Jr., “The Art, Science and Technology of Pharmaceutical Compounding,” (American Pharmaceutical Association, 1999); and Gloria Pickar, “Dosage Calculations,” (Delmar Learning, 1999)). As is well known in the art, adjustments for physiological degradation, systemic versus localized delivery, and rate of new protease synthesis, as well as the age, body weight, general health, sex, diet, time of administration, drug interaction and the severity of the condition may be necessary, and will be ascertainable with routine experimentation by those of ordinary skill in the art.

The pharmaceutical formulations or compositions of the present invention comprise an antibody of the invention in a form suitable for administration to a patient. In the preferred embodiment, the pharmaceutical formulations are in a water soluble form, such as being present as pharmaceutically acceptable salts, which is meant to include both acid and base addition salts. A “pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. A “pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical formulations or compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical formulations may be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The formulations for administration will commonly comprise an antibody of the invention dissolved in a pharmaceutically acceptable carrier or excipient, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (see e.g., “Remington's Pharmaceutical Science,” (15th ed., Mack Publ. Co., Easton Pa., 1980); and Goodman & Gillman, “The Pharmacologial Basis of Therapeutics,” (Hardman et al., eds., TheMcGraw-Hill Companies, Inc., 1996)). The formulations provided herein may also contain more than one active ingredient as necessary for the particular indication being treated, preferably those with complementary activities that do not adversely affect each other. Such molecules are suitably present in combination in amounts that are effective for the purpose intended.

Active ingredients of the above pharmaceutical formulation may be entrapped in microcapsules, in colloidal drug delivery systems (for example, liposome, albumin microspheres, microemulsions, nano-particles and nanocapsules), in macroemulsions, or in sustained-release preparation. Such techniques are known to people skilled in the art (see, e.g., “Remington's Pharmaceutical Science” (15th ed., Mack Publ. Co., Easton Pa., 1980)).

The pharmaceutical formulations or compositions containing anti-IL2 receptor antibodies of the present invention may be administered for therapeutic or prophylactic treatments. In therapeutic applications, compositions are administered to a patient suffering from a disease (e.g., asthma) in an amount sufficient to cure, or at least partially arrest the disease, or otherwise alleviate its symptoms and/or complications. An amount adequate to accomplish this is defined as a “therapeutically effective dose.” The therapeutically effective amount to be used will depend on the specific respiratory disease indication, the type of pharmaceutical formulation, the severity of the disease and the general state of the patient's health. Single or multiple doses of the pharmaceutical formulation may be administered depending on the dosage and frequency as required and tolerated by the patient. In any event, the formulation should provide a sufficient quantity of the active ingredient to effectively treat the patient.

The amount of a pharmaceutical formulation that is capable of preventing or slowing the development of a disease in a mammal is referred to as a “prophylactically effective dose.” The particular dose required for a prophylactic treatment will depend upon the medical condition and history of the mammal, the particular disease being prevented, as well as other factors such as age, weight, gender, administration route, efficiency, etc. Such prophylactic treatments may be used, e.g., in a mammal that has previously had disease to prevent a recurrence of the disease, or in a mammal that is suspected of having a significant likelihood of developing disease.

Dosages and Administration of the Formulation

Generally, pharmaceutical formulations of antibodies may be prepared for storage by mixing the antibodies having the desired degree of purity with optional physiologically acceptable carriers, excipients, or stabilizers, in the form of lyophilized or aqueous solutions. Acceptable carriers, excipients or stabilizers are nontoxic to recipients at the dosages and concentrations employed, and include buffers such as phosphate, citrate, and other organic acids; antioxidants, preservatives, low molecular weight polypeptides, proteins, hydrophilic polymers, amino acids, carbohydrates, chelating agents, sugar, and other standard ingredients known to people skilled in the art (“Remington's Pharmaceutical Science” supra).

The daclizumab formulation described herein for in vivo administration is usually stored at 2° to 8° C. The formulations often contain no preservatives and should be used within 4, 12 or 24 hours of withdrawal from the vial and dilution into saline. The formulation is preferably administered intravenously or subcutaneously with or without filtration. In one embodiment, the anti-IL2 receptor antibody formulation may be stored in a stable lyophilized form according to the methods described in U.S. patent application Ser. No. 10/206,469, filed Jul. 25, 2002, which is hereby incorporated by reference herein in its entirety.

Preferably, the humanized anti-IL-2 receptor antibody, daclizumab is stored in a single-use glass vial containing 5.0 mL of daclizumab at a concentration of 5.0 mg/mL in sterile saline buffer. However, concentrations from 1 to 10 mg/mL (e.g., 1, 2, 5 or 10), 20 to 50 mg/mL (e.g., 20, 30, 40 or 50) or 60 to 100 mg/mL (e.g., 60, 70, 80, 90 or 100) are also encompassed by the present invention. In a preferred formulation for storage, the formulation comprises 5 mg/mL of the antibody, 3.6 mg/mL sodium phosphate monobasic monohydrate, 11 mg/mL sodium phosphate dibasic heptahydrate, 4.6 mg/mL sodium chloride, 0.2 mg/mL polysorbate 80. The formulation may further comprise hydrochloric acid or sodium hydroxide to adjust the pH of the formulation to about 6.9.

In one preferred embodiment, daclizumab may be prepared as a stable liquid formulation as described in U.S. patent application Ser. No. 10/291,528, filed Nov. 8, 2002 (U.S. published application Ser. No. 2003/0138417 A1, published Jul. 24, 2003) which is hereby incorporated by reference herein, in its entirety. This stable liquid formulation is particularly useful for subcutaneous administration of daclizumab, and may be used in the method for treating respiratory disease of the present invention. In a preferred embodiment, the stable liquid formulation of daclizumab comprises about 100 mg/ml daclizumab, about 20-60 mM succinate buffer (or about 20-70 mM histidine buffer) having pH from about 5.5 to about 6.5, about 0.01% -0.1% polysorbate, and a tonicity buffer that contributes to isotonicity of the formulation (e.g. about 75-150 mM NaCl, or about 1-100 mM MgCl₂).

Therapeutic antibodies prepared in a pharmaceutical formulation may be administered by any suitable route including oral, rectal, nasal, topical (including transdermal, aerosol, buccal and sublingual), parenteral (including subcutaneous, intramuscular, intravenous and intradermal) or by inhalation therapy. In one embodiment, the formulation may be administered using a needle-free air-pressure shot. It will also be appreciated that the preferred route may vary with the condition and age of the recipient. Preferably, the pharmaceutical formulation is delivered parenterally, for example, intravenously by bolus injection, so that a therapeutically effective amount of said formulation is delivered via systemic absorption and circulation.

The therapeutically effective amount of the formulation depends on the severity of the specific respiratory disease indication (e.g. severe chronic asthma), the patient's clinical history and response, and the discretion of the attending physician. The formulation may be administered to the patient at one time or over a series of treatments. An initial candidate dosage may be administered to a patient and the proper dosage and treatment regimen established by monitoring the progress of this patient using conventional techniques well known to those of ordinary skill in the art.

The amount of active ingredients that may be combined with the carrier materials to produce a single dosage form will vary depending upon the subject treated and the particular mode of administration. It will be understood, however, that the specific dose level for any particular patient will depend upon a variety of factors, including the activity of the specific formulation employed, the age, body weight, general health, sex, diet, time of administration, route of administration, rate of excretion, drug combination and the severity of the particular disease undergoing therapy, and can be determined by those skilled in the art.

In particular, an exemplary effective dose for the treatment of asthma is between about 0.001 mg/kg (i.e. milligram per kilogram body weight) to about 100 mg/kg, preferably between about 0.001 mg/kg to about 10 mg/kg, and more preferably about 0.005 mg/kg to about 0.100 mg/kg. Preferred dose levels include about 0.001 mg/kg, about 0.005 mg/kg, about 0.0075 mg/kg, about 0.010 mg/kg, about 0.015 mg/kg, about 0.020 mg/kg, about 0.030 mg/kg, about 0.045 mg/ kg, about 0.050 mg/kg, about 0.060 mg/kg, about 0.070 mg/kg, about 0.080 mg/kg, and about 0.1 mg/kg. The preferred dose can be equal to or less than about 0.5 mg/kg, 1 mg/kg, 2 mg/kg, 3 mg/kg, 4 mg/kg, or 5 mg/kg. The preferred dose can be within a range of any two of the above-indicated dose levels.

“Fixed dose” formulations of anti-IL2 receptor antibodies may also be prepared and administered to patients. For example, a pre-filled 1 ml syringe of a 100 or 200 mg/ml daclizumab formulation may be administered to all asthma patients regardless of patient weight. Given a typical adult patient population of weight between 50 and 100 kg, a 100 mg fixed dose delivers between 1 mg/kg and 2 mg/kg. Fixed dose formulations minimize possible dosage errors in administration and may be particularly preferred for treatment of asthma where a dose may be administered by the patient to himself.

Generally, higher dosages (e.g. from 0.1 up to about 100 mg per patient per day) of therapeutic antibodies may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, see e.g., “Remington's Pharmaceutical Science,” and Goodman and Gillman, “The Pharmacologial Basis of Therapeutics,” supra.

Treatment Regimen

Depending on the progress in treatment and the physical conditions of the patients, the regimen of the treatment of asthma can vary significantly. Typically, a patient is administered at least a single dose of pharmaceutical formulation comprising any one of the antibodies described herein, which is named as “the initial dose” or “the initial administering or administration” or “the loading dose” if there are any additional doses (“maintenance dose”) follow. The antibody drug can be administered once or multiple times at a frequency of e.g., 1, 2, 3, or 4 times per day, weekly, bi-weekly, every 6 weeks, or monthly, or every 2, 3, or 6 months. The duration of the treatment of one treatment course should last for at least one or two days, such as, one to several (2, 3, 4, 5, or 6) days, weeks, months or years, or indefinite, depending upon the nature and severity of the disease. The duration of the treatment is calculated as the period from the initial administration of the antibodies to the last administration of the antibodies. The patient may receive 2, 3, 4 or more courses of treatment. The frequency of the administration can be adjusted according to the improvement progress of the patients. A preferred loading dose is about 2 mg/kg. A preferred maintenance dose, subsequent to the loading dose, is about 1 mg/kg. In a preferred dosing schedule, the loading dose is administered over a 30-minute period, and each maintenance dose is administered over a 15-minute period.

To reduce the infusion-related symptoms, the pharmaceutical formulation comprising anti-IL-2 receptor antibodies may also be used as separately administered formulations given in conjunction with other agents. Typically, these agents include methyprednisolone, hydrocortisone, ondansetron, acetaminophen, and numerous additional agents that have the similar functions and are well-known to those skilled in the art. These other agents can be administered by any suitable route including oral, rectal, nasal, topical, parental (including subcutaneous, intramuscular, intravenous and intradermal), or by inhalation therapy.

The dose levels of these agents are also known in the art, for example, from 1 mg to 100 g per patient. Exemplary doses include 10-50 mg, 60-200 mg, or 200-500 mg for methyprednisolone, hydrocortisone and ondansetron; and 100-500 mg, 600-1000 mg, 1-5 g for acetaminophen. Single or multiple additional immunomodulating agents can be administered to the patients, for example, at least about 1, 2, 3, 4, 5, 6, 7, 8, 10, 12, 14, 20, 24, 36 hours or 2, 3, 4, 5, 7, 10, 20, 40, or 60 days, prior to or/and after the initial or/and each administering of the pharmaceutical formulation of anti-IL-2 receptor antibodies.

In one embodiment, the method of treatment of the present invention further comprises administering a concomitant medication for the target disease indication. For example, concomitant asthma medications (for both chronic and acute) that may be used with the method of the present invention include but are not limited to: inhaled and oral steroids (e.g. beclomethasone, budesonide, flunisolide, fluticasone, triamcinolone, mometasone and acetonide); systemic corticosteroids (e.g. methylprednisolone, prednisolone, prednisone, dexamethasone, and deflazacort); inhaled or oral β2 agonists (e.g. salmeterol, formoterol, bitolterol, pirbuterol, terbutaline, bambuterol and albuterol); cromolyn and nedocromil; anti-allergic opthalmic medications (e.g. dexamethasone); methylxanthines (e,g. theophylline and mepyramine-theophylline acetate); leukotriene modifying agents (e.g. zafirlukast, zileuton, montekulast and pranlukast); anticholinergics (e.g. ipatropium bromide); other therapeutic antibodies (e.g. antibodies directed against intracellular adhesion molecules or IgE); thromboxane A2 synthetase inhibitors; thromboxane prostanoid receptor antagonists; other eicosanoid modifiers (e.g. alprostadil vs. PGE1, dinoprostone vs. PGE2, epoprostenol vs. prostacyclin and PGI2 analogues (e.g. PG12 beraprost), seratrodast, ozagrel, phosphodiesterase 4 isoenzyme inhibitors, thromboxane A2 synthetase inhibitors (e.g. azelastine); ditec (low dose disodium cromoglycate and fenoterol); platelet activating factor receptor antagonists; antihistamines; anti-thromboxane A2; antibradykinins (e.g. icatibant); agents that inhibit activated eosinophils and T-cell recruitment (e.g. ketotifen), IL-13 blockers (e.g. soluble IL-13 receptor fragments), IL-4 blockers (e.g. soluble IL-4 receptor fragments); ligands that bind and block the activity of IL-13 or IL-4, and xanthine derivatives (e.g. pentoxifyolline).

The following examples are offered by way of illustration and not by way of limitation. The disclosure of all citations in the specification is expressly incorporated herein by reference for all purposes.

### EXAMPLES

### Example 1

### A Phase II, Randomized, Double-Blind, Placebo-Controlled, Parallel-Group Study of Daclizumab in Patients with Chronic, Persistent Asthma

This example describes the design, execution and results of a Phase II, dose-escalation, pilot study demonstrating the efficacy of a method of using daclizumab for treating patients with chronic, persistent asthma.

A. Overview of the Study

1. Objectives

The primary objectives of the study are to evaluate the safety, tolerability, and preliminary activity of daclizumab in adult patients with chronic, persistent asthma who are sub-optimally controlled on inhaled corticosteroids.

2. General Study Design

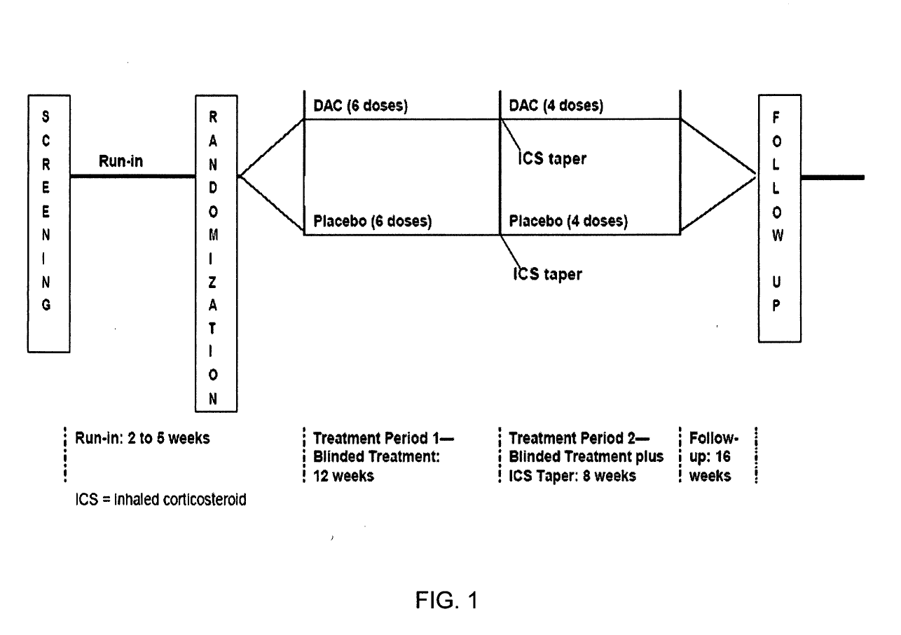

The study was a randomized, multicenter, double-blind, placebo-controlled, parallel-group study of daclizumab in the treatment of patients with chronic, persistent asthma, who are sub-optimally controlled on inhaled corticosteroids (equivalent of >1200 μg daily inhaled triamcinolone). The general design of the study is illustrated by scheme shown in FIG. 1.

A screening visit was followed by a run-in period of up to 5 weeks, with randomization at baseline to active drug or placebo (3:1). Study visits occurred at screening (Visit 1), during the run-in period (up to 4 visits), every 2 weeks during the Treatment Period 1 (Days 0 to 84, 6 visits), every 2 weeks through Treatment Period 2 (Days 85 to 140 steroid taper, 4 visits), and then every 2 to 4 weeks during the Follow-up Phase (Days 141 through 239, 5 visits). All study visits, dosing and assessments were conducted ±3 days of the designated study day.

3. Selection of Patient Population

Eligible for enrollment in the study are nonsmoking male and female asthmatic patients aged 18 to 70 years who demonstrate a forced expiratory volume in 1 second (FEV₁) between 50% to 80% predicted and reversibility of >12% with β₂-agonist, despite requiring daily doses of >1200 μg inhaled triamcinolone acetonide (triamcinolone) or its equivalent for >3 months prior to enrollment. In order to be randomized, patients must demonstrate a requirement for inhaled corticosteroids, an FEV₁>90% of Screening (absolute value), and an FEV₁>50% predicted during the Run-in period. Specifically, an increase of ≧12% and 200 mL in absolute FEV₁ following 2 to 4 inhalations of albuterol MDI demonstrated at Screening or during the Run-in phase was necessary to qualify a patient for randomization. A sample size 120 randomized patients (90 active, 30 placebo) was used.

Patients who meet all of the inclusion criteria and none of the exclusion criteria listed in Table 1 were considered for enrollment into this study.

TABLE 1
Inclusion and Exclusion Criteria
Inclusion Criteria

Male or female patients age 18 to 70 years
History of asthma for ≧6 months
History of chronic persistent asthma requiring ≧1200 μg inhaled triamcinolone daily or equivalent dose
of inhaled corticosteroid for ≧3 months prior to enrollment (see, Table 2 below)
FEV₁ 50% to 80% of predicted at the time of enrollment. This value was designated as the prestudy
baseline. An increase of ≧12% and 200 ML in absolute FEV1 following 2 to 4 inhalations of albuterol
MDI demonstrated at Screening or during the Run-in phase qualified a patient for randomization.
Women of childbearing potential who provided negative serum pregnancy test at screening and a
negative urine pregnancy test within 24 hours prior to the first dose of blinded study drug. Both men
and women required to document use of double barrier contraception during the study and for 4 months
after the final infusion of study drug
Patients who provided informed consent

Exclusion Criteria

Body weight less than 70% ideal weight for height and sex.
Patients who had received any experimental drug treatment within 30 days of study enrollment
Patients who had received treatment with any murine, chimeric, humanized antibody or IV IG
within 90 days of study enrollment
Patients who had received treatment with cyclosporine; methotrexate; or
troleandomycin/methylprednisolone within 60 days of enrollment
Patients who had donated >500 mL, of blood in the 8 weeks prior to study enrollment
Patients with ≧10 pack years of smoking history or smoking within 12 months of study enrollment
Patients with an upper or lower respiratory tract infection within 14 days of study enrollment
Patients who had been vaccinated within 6 weeks of study entry (Patients who have received
influenza vaccination will be permitted entry at 2 weeks following vaccination.)
Patients receiving antibiotic treatment for acute or chronic sinusitis
Patients with any significant organ dysfunction, including pulmonary (other than asthma), cardiac,
liver, renal, central nervous system, vascular, gastrointestinal, endocrine, or metabolic
Patients with creatinine ≧1.6 mg/dL, alanine aminotransferase (ALT) or aspartate aminotransferase
(AST) ≧2.5x the upper limit of normal
Patients with history of myocardial infarction, congestive heart failure, or arrhythmia ≦6 months of
study enrollment
Patients receiving beta-blocker therapy
Patients with preexisting evidence of infection with human immunodeficiency virus or presence of
hepatitis B surface antigen or positive hepatitis C serology
Hemoglobin <11 g/dL, platelets <100,000/mm³, or neutrophils <1500 cells/mm³
Patients who had had major surgery within 30 days of study enrollment
Patients who had used oral or parenteral corticosteroids within 30 days of study enrollment
Patients who had been hospitalized for asthma within 60 days of study enrollment
Patients receiving allergen immunotherapy or who have received submaintenance allergen
immunotherapy within 6 months of enrollment or who had received maintenance allergen
immunotherapy within 2 years of enrollment
Pregnant or lactating females
Patients with a hypersensitivity to heparin
Patients with a hypersensitivity to daclizumab or any of the excipients in the formulation
Serious infection requiring intravenous antibiotics or hospitalization within 60 days of enrollment
Prior malignancy within 5 years or concurrent malignancy (excluding non-melanoma skin carcinoma, or
in situ carcinoma of the cervix that has been adequately treated)
Patients unable to comply with the study protocol or with significant disabilities or incapacities
Patients who had had varicella(chickenpox), herpes zoster shingles), or other serious viral infections
within 6 weeks of study enrollment.
Patients who had had exposure to chickenpox within 21 days of study enrollment.

TABLE 2
Daily Inhaled Steroid Equivalent Doses Required for Inclusion

| Drug | Dose |
|---|---|
| Fluticasone MDI | ≧330 μg (≧3 110 μg MDI puffs) |
| Beclomethasone | ≧588 μg (≧14 42 μg puffs) |
| Budesonide | ≧400 μg (>2 inhalations) |
| Flunisolide | ≧1200 μg (≧5 puffs) |
| Triamcinolone | ≧1200 μg (≧12 puffs) |
| Fluticasone DPI | ≧4 inhalations 100 μg DPI or ≧2 inhalations 250 μg |
| (including Advair | DPI or ≧1 inhalation 500 μg DPI |
| disckus) | |

Patients with the following were considered for enrollment:
a. Patients on a stable dose of nasal corticosteroids, nasal cromolyn, topical antiallergic ophthalmic medications or corticosteroid creams or ointments for >30 days prior to enrollment
b. Patients who have been on treatment with theophylline, salmeterol, oral albuterol, Advair (fluticasone and salmeterol oral inhalation), cromolyn, nedocromil, or leukotriene modifying agents (These medications must be discontinued at enrollment).
c. Patients using short-acting inhaled or nebulized β₂-agonist as needed.

Patients who signed the informed consent were screened. Those patients meeting the criteria for randomization were assigned to one of two study arms (daclizumab or placebo). The NRS center communicated the patient's randomization number to the designated study center pharmacist. The first day of blinded treatment (daclizumab or placebo) was designated Day 0.

4. Drug Preparation, Dosage and Delivery

The daclizumab used in the study was obtained as ZENEPAX®, the commercially available, FDA-approved preparation of daclizumab. Placebo was the comparative drug. Daclizumab was supplied in vials containing 25 mg of daclizumab in 5 ml, of solution. Each milliliter of solution contained 5 mg daclizumab and 3.6 mg sodium phosphate monobasic monohydrate, 11 mg sodium phosphate dibasic heptahydrate, 4.6 mg sodium chloride, 0.2 mg polysorbate 80, and may contain hydrochloric acid or sodium hydroxide to adjust the pH to 6.9. No preservatives were added.

The placebo consisted of all the components provided in the active formulation, minus the active ingredient (daclizumab). The fill size for the placebo vial was 5 mL.

The doses used were 2 mg/kg for the loading dose, and 1 mg/kg for subsequent doses. The study medication was administered intravenously after the dose had been diluted with 50 ml, of normal saline (0.9%) in a minibag. The 2 mg/kg loading dose was infused over a 30-minute period. All subsequent 1 mg/kg doses were infused over a 15-minute period. All infusions were administered using an infusion pump. Daclizumab is not for direct injection and must be diluted before use.

Patients remained under observation for at least 2 hours following the completion of the first study drug infusion (Day 0) and at least one hour after all subsequent infusions.

Daclizumab and placebo were stored under controlled, refrigerated conditions at 2° to 8° C. The formulation contains no preservative and was to be used within 24 hours of withdrawal from the vial. Once an infusion is prepared, it should be administered intravenously within 4 hours. If the preparation is not used immediately, it should be refrigerated between 2° to 8° C. for up to 24 hours, or held up to 4 hours at room temperature. After that time, the prepared solution should be discarded.

5. Study Dose Interval Schedule, Dose Escalation and Follow Up

The study treatment dosing regimen is summarized in Table 3 below.

TABLE 3
Treatment Dosing Regimen

| | Treatment Period 1¹ | | | | | | Treatment Period 2² | | | | Follow-up | | | | | |
|---|---|---|---|---|---|---|---|---|---|---|---|---|---|---|---|---|
| | | Day | Day | Day | Day | Day | Day | Day | Day | Day | Day | Day | Day | Day | Day | Day |
| Tx | Day 0 | 14 | 28 | 42 | 56 | 70 | 84 | 98 | 112 | 126 | 140 | 154 | 168 | 182 | 210 | 238 |
| Dac | x³ | x | x | x | x | x | x | x | x | x | N/A | N/A | N/A | N/A | N/A | N/A |
| Pbo | x³ | x | x | x | x | x | x | x | x | x | N/A | N/A | N/A | N/A | N/A | N/A |

¹All patients will receive inhaled triamcinolone at prerandomization dose (Days 0-83), in addition to blinded treatment.
²All patients will undergo 25% reduction of prerandomization triamcinolone dose at 2-week intervals, beginning on day 84, in addition to receiving blinded treatment.
³2 mg/kg loading dose on day 0, infused over a 30-minute period, followed by 1 mg/kg dose, infused over a 15-minute period, on all other dosing days.
Dac = daclizumab,
N/A = not applicable-no study drug infused,
Pbo = placebo

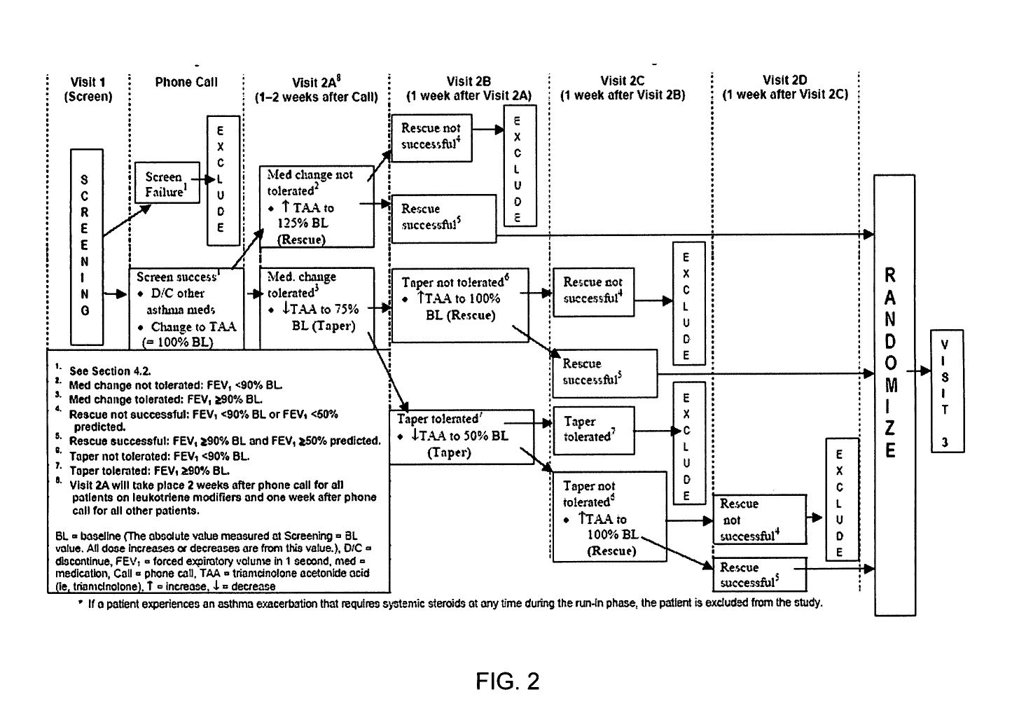

The study included a Run-in period from days −28 to −1. The purpose of the Run-in phase was to verify a requirement for inhaled corticosteroids in the study patients. During the Run-In period, patients underwent a inhaled corticosteroid titration process as summarized by the schematic shown in FIG. 2. Patients were switched to the equivalent dose of inhaled triamcinolone and discontinued all other concomitant asthma controller medications at the beginning of the Run-in. Patients who discontinued leukotriene modifiers waited 2 weeks after entering the run-in to return for Visit 2A. All other patients waited only one week after discontinuing all other asthma controller medications to return for Visit 2A. To be randomized, patients were required to demonstrate a requirement for inhaled corticosteroids, an FEV₁>90% of Screening (absolute value), and an FEV₁>50% predicted during the Run-in. Patients who had an asthma exacerbation requiring systemic corticosteroids were not randomized and were discontinued from the study.

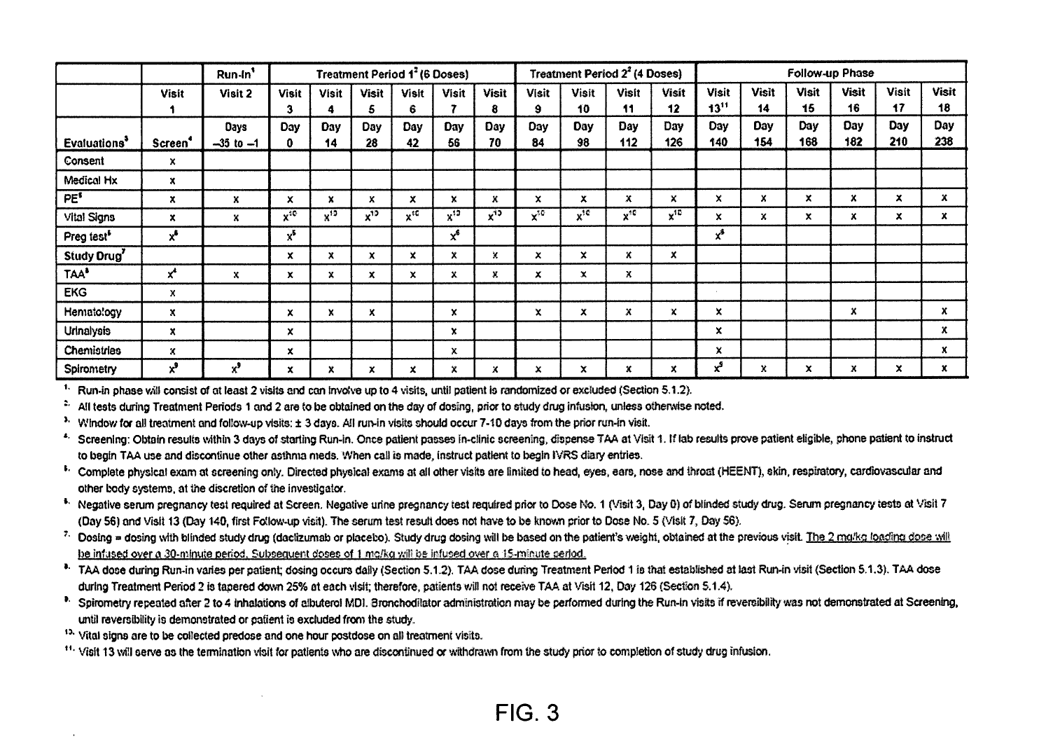

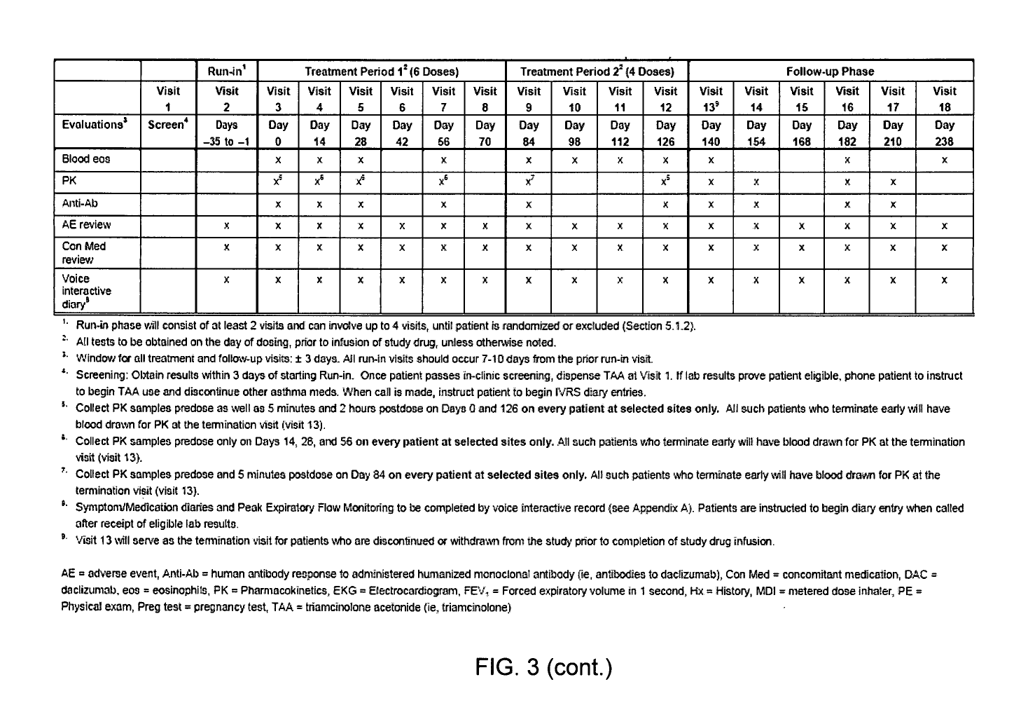

The dose interval and escalation schedule and associated patient assessments is summarized in the table shown in FIG. 3. The schedule was divided into two treatment periods: (1) Treatment Period 1 (blinded treatment, Days 0 to 84): loading dose of 2 mg/kg daclizumab or placebo on Day 0, infused over a 30-minute period, followed by subsequent doses of 1 mg/kg, infused over a 15-minute period, on Days 14, 28, 42, 56, and 70; (2) Treatment Period 2 (blinded treatment plus steroid taper, Days 85 to 140): 1 mg/kg of daclizumab or placebo on Days 84, 98, 112, and 126, infused over one 15-minute period.

During Treatment Period 1 eligible patients were randomized (3:1, active: placebo) to receive daclizumab or placebo infusions on Days 0, 14, 28, 42, 56, and 70 while maintaining the prerandomization baseline dose of inhaled triamcinolone. For patients receiving daclizumab, a loading dose of 2 mg/kg daclizumab, infused over a 30-minute period, will be administered on Day 0, followed by doses of 1 mg/kg, infused over a 15-minute period, on subsequent treatment days. Patients received the first dose of blinded study drug no later than 7 days from the final Run-in visit. After randomization, patients were seen every 2 weeks for assessment and dosing.

During Treatment Period 2 (i.e. following Treatment Period 1), patients had their inhaled triamcinolone (TAA) dose reduced by 25% of the dose on which they completed Treatment Period 1 at two-week intervals (Days 84, 98, and 112) until they had completely eliminated inhaled steroids (Day 126) while receiving infusions of daclizumab 1 mg/kg or placebo (Days 84, 98, 112, and 126), infused over a 15-minute period. Patients were seen every two weeks for assessments and dosing.

Patients meeting the following criteria for treatment cessation at the designated time point of the study were withdrawn from further dosing of study drug and entered into the Follow-up phase: (1) experienced more than one asthma exacerbation requiring systemic steroid rescue during Treatment Period 1; (2) experienced a single asthma exacerbation requiring systemic steroid rescue during Treatment Period 2; (3) required >60 mg daily prednisone during any asthma exacerbation; (4) required >14 days of treatment with prednisone during an asthma exacerbation; or (5) required overnight hospitalization for an asthma exacerbation (i.e. required >24 hour hospital stay).

In the Follow-up Phase (Days 140 to 238), patients were monitored for 16 weeks off study drug and evaluated on Days 140, 154, 168, 182, 210, and 238.

All individuals at the study sites were blinded to treatment. These individuals include the investigator, study coordinator, patient, and other study personnel. The PDL medical monitor and CRA also remained blinded to treatment. A designated PDL safety monitor not involved with the conduct of study reviewed all Grade 2 or higher (i.e., moderate, severe or life-threatening) events, whether related or unrelated to administration of study drug, on a monthly basis. This individual will be unblinded to treatment.

6. Compliance, Safety and Pharmacodynamic Measurements

Study medication was administered under the supervision of study personnel. Compliance was assured by having the investigator or qualified designee administer each infusion of blinded study drug. Use of inhaled triamcinolone was assessed by study personnel on a patient-by-patient basis and by recording all triamcinolone MDI canisters dispensed to the patients.

Safety measurements made during the course of the study included vital signs, nurse/physician observations, assessment of adverse events, and safety laboratory profiles.

Pharmacodynamics were measured for all study patients via measurements of whole blood eosinophils.

Serum daclizumab concentrations were obtained throughout the study and used to provide a limited pharmacokinetic profile of daclizumab over time. Serum samples were collected from every patient at selected sites only prior to dosing with study medication and at various time points after dosing (See Table 3).

Immunogenicity was assessed in each patient by analysis of antibodies against daclizumab (Anti-Ab) in serum samples.

7. Efficacy Measurements

The primary efficacy parameter used to assess daclizumab was pulmonary function (spirometry) as assessed by the percent change from baseline in FEV₁, during Treatment Period 1 (Day 0 to Day 84).

The secondary efficacy parameters included: (1) proportion of patients who experience asthma exacerbation during Treatment Period 1 (Days 0 to 84); (2) percent change in AM PEFR and PM PEFR during Treatment Periods 1 (Days 0 to 84) and 2 (Days 85 to 140); (3) change in rescue medication (β₂-agonist MDI) use, daytime and nighttime asthma symptoms, and in asthma-free days as recorded in the IVRS during Treatment Periods 1 and 2; (4) proportion of patients who withdraw from the study during Treatment Periods 1 and 2; (5) time to asthma exacerbation during Treatment Periods 1 and 2; and (6) percent change in FEV₁ during Treatment Period 2 and Follow-up.

Spirometry Measurements

Spirometry measurements were recorded according to the schedule in FIG. 3. All spirometry measurements were performed in accordance with the American Thoracic Society Guidelines. The predicted values used were consistent throughout the study. Spirometry values were not adjusted for race. The best FEV₁ of three efforts was recorded. Spirometry consisted of forced vital capacity (FVC), FEV₁, and FEV₁/FVC, forced expiratory flow during the middle half of the FVC (FEF 25-75). Short-acting inhaled β₂-agonist agents were withheld for 6 hours prior to the spirometry. Spirometry measurements for each visit were recorded in the patient's Case Report Form.

Treatment of Asthma Exacerbations

An asthma exacerbation was defined as increased cough, chest tightness, or wheezing in association with 1 or more of the following: (1) rescue albuterol use of ≧8 puffs per 24 hours over baseline use for a period of 48 hours; (2) rescue albuterol use of ≧16 puffs per 24 hours for a period of 48 hours; (3) peak expiratory flow (PEF)<65% of reference level despite 60 minutes of rescue treatment; (4) symptoms despite 60 minutes of rescue treatment (defined as 2 to 4 puffs of albuterol every 20 minutes for up to 1 hour); and/or (5) requirement for systemic (oral or injectable) corticosteroids

Study investigators assessed the need for systemic steroid rescue. In addition to following the criteria above, the study investigator was also allowed to prescribe systemic steroids for asthma exacerbation according to his or her discretion. The reason for any use of systemic corticosteroids was documented on the patient's Case Report Form (CRF). Patients were advised to call the study center if they experienced an asthma exacerbation (according to the above criteria). Patients with exacerbations requiring treatment with oral corticosteroids were permitted a prednisone burst of up to 60 mg per day for up to 14 days. Any patients who met the criteria for treatment cessation were discontinued from further dosing and entered the Follow-up phase. Visit 13 served as the termination visit. Only those exacerbations that required the use of systemic steroid rescue were used to determine treatment cessation.

Patients who experienced their first asthma exacerbation requiring systemic corticosteroid rescue during Treatment Period 1 also had their dose of inhaled triamcinolone increased by 25% from the prerandomization baseline and continued on this dose until the end of Treatment Period 1.

Asthma Symptom/Medication Diary Record

The symptoms of asthma, medication use, and peak flow recording were assessed using each patient's daily diary recording (by interactive voice response system) during the study. The IVRS was considered a source document; this type of diary has been previously validated for use in asthma clinical trials.

The daytime mean symptom scale uses a range of response categories for each question from 0 to 6, indicating the least to the most asthma symptoms. The nocturnal diary scale uses response categories ranging from 0 (indicating no awakening with asthma symptoms) to 3 (indicating awake all night). Daily daytime scale scores were computed as the mean total of the 4 questions on the daytime symptom scale. An overall diary score for the week was computed as the mean of at least 4 of 7 daily daytime scale scores. Weekly mean scores for the nocturnal diary scale were computed in a similar manner. A decrease in the weekly score for the daytime and nocturnal scales indicates an improvement in asthma symptoms. The change from baseline in the asthma scale scores was computed as the difference between the mean score from the last week of the pre-treatment run-in period (Days −7 to −1) and the last week of Treatment Period 1 (Days 77 through 84) and the last week of Treatment Period 2 (Days 134 to 140).

Asthma-free days were defined as those days in which both the diurnal and nocturnal diaries indicate no symptoms. The method by which this variable was analyzed is dependent upon the distribution of missing diary entries. If there were few missing data, changes in mean asthma-free days will be analyzed. If patients were inconsistent in reporting their asthma symptoms, then the proportion of patients who experienced asthma-free days was compared by treatment group.

Rescue use of β₂-agonist MDI was recorded in the daytime and nocturnal symptom diaries. Patients were instructed to record the number of actuations of β₂-agonist from a MDI that were used during the day in the daytime diary recording and the number of actuations of β₂-agonist MDI that were used after going to sleep for the night in the nocturnal diary recording. The mean daily use of β₂-agonist MDI was computed for daily and nocturnal use for each week of diary recording. The change from baseline in the use of β₂-agonist rescue medication was computed as the difference between the mean daily score from the week of the pretreatment run-in period (Days−7 to−1) and the last week of Treatment Period 1 (Days 77 to 84) and the last week of Treatment Period 2 (Days 134 to 140).

Peak Expiratory Flow Monitoring

During the screening visit, patients were instructed in the use of the mini-Wright peak flow meter. Patients measured and recorded the best of 3 PEFRs in the daytime diary recording at night before going to sleep and in the nocturnal diary recording on arising in the morning prior to taking any medications. An overall mean daily nighttime and morning peak flow was computed for each week. The change from baseline for nighttime and morning peak flow was computed as the difference in the mean daily PEFR from the last week of the pretreatment run-in period and the last week of Treatment Period 1 (Days 77 through 84) and Treatment Period 2 (Days 134 to 140).

8. Statistical Methods

Descriptive statistic analyses for each treatment group were carried out for the demographic and baseline variables. Continuous variables, such as age, disease duration, and symptom scores, were assessed by t-tests or equivalent nonparametric tests. Categorical variables were assessed by chi-square or Fisher's exact tests, as appropriate.

Evidence of preliminary efficacy (preliminary efficacy: change from baseline in FEV₁, secondary efficacy: symptom scores, β₂-agonist rescue medication use, number of asthma exacerbations, and mean daily AM peak expiratory flow rate) was presented descriptively for placebo vs. daclizumab. Within-group changes were evaluated by paired t-tests or Wilcoxon Signed Rank tests. Between-group statistical significance was determined by t-tests or Wilcoxon-Mann-Whitney tests. Assessment of time-to-event variables employed Kaplan-Meier and log rank methods.

B. Detailed Study Protocols

A more detailed description of the methods and protocols used in this Phase II study are disclosed in U.S. provisional application 60/552,974, filed Mar. 12, 2004, each of which is hereby incorporated by reference herein for all purposes.

A complete description of the Phase II protocol is found in Protein Design Lab Protocol No. DAC-1003 (“A Phase II, Randomized, Double-Blind, Placebo-Controlled, parallel-Group Study of Daclizumab in Patients with Chronic, Persistent Asthma”), Daclizumab, dated Mar. 27, 2001; Amendment A: Jul. 16, 2001; Amendment B: Aug. 24, 2001; Amendment C: Apr. 15, 2002; Amendment D: Jul. 8, 2002; Amendment E: Aug. 28, 2002 (which is herein incorporated by reference in its entirety).

TABLE 4
Abbreviations Used in Phase II Protocols

| Absolute | |
|---|---|
| baseline | |
| (value) | Absolute value of FEV₁, measured at the Screening Visit |
| AE | Adverse event (experience) |
| FEV₁ | Forced expiratory volume in one second |
| HEENT | Head, eyes, ears, nose, and throat |
| IVIG | Intravenous immunoglobulin |
| MDI | Metered dose inhaler |
| PEF | Peak expiratory flow |
| PEFR | Peak expiratory flow rate |
| SAE | Serious adverse event (experience) |
| TAA | Triamcinolone acetonide (also referred to as Triamcinolone) |

C. Detailed Study Results

1. Patient Disposition and Baseline Characteristics

Tables 5-8 list the patient disposition, demographics and baseline characteristics prior to treatment with daclizumab.

TABLE 5
Patient Disposition, Pre-randomization

| | Total |
|---|---|
| Description | N (%) |
| Patients Enrolled | 208 |
| Drop Outs Pre-Randomization | 92 |
| Exacerbation | 3 (3.3) |
| Run-in Failure | 61 (66.3) |
| Non-compliance | 3 (3.3) |
| Protocol Violation | 2 (2.2) |
| Investigator judgment | 3 (3.3) |
| Patient Decision | 13 (14.1) |
| Adverse Event | 1 (1.1) |
| Other | 6 (6.5) |
| Patients to Be Randomized | 116 |

TABLE 6
Patient Disposition, Post-randomization

| | Daclizumab | Placebo | Total |
|---|---|---|---|
| Description | N (%) | N (%) | N (%) |
| Patients Randomized, n (row %) | 89 (76.7) | 27 (23.3) | 116 (100) |
| Drop Outs Pre-Dosing* | 2 (2.2) | 0 (0.0) | 2 (1.7) |
| Investigator judgment | 1 (1.1) | 0 (0.0) | 1 (0.9) |
| Patient Decision | 1 (1.1) | 0 (0.0) | 1 (0.9) |
| Drop Outs Post-Dosing* | 11 (12.4) | 4 (14.8) | 15 (12.9) |
| Adverse Event | 1 (1.1) | 0 (0.0) | 1 (0.9) |
| Non-compliance | 1 (1.1) | 0 (0.0) | 1 (0.9) |
| Withdrew | 7 (7.9) | 2 (7.4) | 9 (7.8) |
| Investigator judgment | 1 (1.1) | 0 (0.0) | 1 (0.9) |
| Other | 1 (1.1) | 2 (7.4) | 3 (2.6) |
| Patients Completed Study | 76 (85.4) | 23 (85.2) | 99 (85.3) |

TABLE 7
Patient demographics

| | Daclizumab | Placebo | |
|---|---|---|---|
| | n = 88 (77%) | n = 27 (23%) | p-value |
| Female (n (%)) | 56 (63.6%) | 19 (70.4%) | 0.68 |
| Caucasian (n (%)) | 75 (85.2%) | 20 (74.1%) | 0.30 |
| Age (yr) | | | |
| Mean (SD) | 43.2 (12.5) | 41.0 (12.4) | 0.42 |
| Median | 44.2 | 37.6 | |
| (Min, Max) | (18.6, 73.3) | (23.4, 70.9) | |

TABLE 8
Baseline Characteristics (1)

| | Daclizumab | Placebo | |
|---|---|---|---|
| Mean (SD) | n = 88 (77%) | n = 27 (23%) | p-value |
| Disease Duration (yr) | 22.6 (13.6) | 24.9 (14.0) | 0.47 |
| Years of Inhaled | 5.4 (6.1) | 4.6 (5.3) | 0.53 |
| Corticosteroid Use | | | |
| Prerandomization Dose | 2089 (769) | 2111 (729) | 0.89 |
| of Triamcinolone (μg) | | | |
| FEV1 (L/sec) | 2.3 (0.7) | 2.2 (0.5) | 0.46 |
| FEV1 % Predicted | 68.8 (11.3) | 68.0 (11.1) | 0.75 |
| Daily Symptom Score | 7.8 (4.1) | 9.4 (4.2) | 0.10 |
| Daily B2 Agonist | 3.9 (3.2) | 4.6 (3.1) | 0.32 |
| Rescue Medication Use | | | |
| (no. of puffs) | | | |
| Daily Morning PEFR | 370.4 (96.1) | 344.9 (78.9) | 0.19 |
| (L/Min) | | | |
| Whole Blood | 2.6 (2.1) | 2.4 (1.9) | 0.69 |
| Eosinophils (%) | | | |
| Reversibility | 21.9 (9.8) | 21.0 (9.4) | 0.68 |
| % Change | | | |

2. Protocol-Specified Analyses

Tables 9-17 list protocol-specified analyses of patient data obtained during the Treatment Period 1 (Day 0-Day 84).

TABLE 9
FEV₁ (Day 0-Day 84)

| | Daclizumab | Placebo |
|---|---|---|
| | n = 88 (77%) | n = 27 (23%) |
| Baseline (L/sec) | | |
| Mean (s.e.) | 2.34 (0.07) | 2.25 (0.1) |
| N | 88 | 27 |
| Day 84 (L/sec) | | |
| Mean (s.e.) | 2.43 (0.08) | 2.20 (0.1) |
| N | 76 | 26 |
| % Change from Baseline | | |
| Mean (s.e.) | 4.40 (1.80) | −1.52 (2.39) |
| N | 76 | 26 |
| Within-Group p-value | 0.02 | 0.53 |

| | |
|---|---|
| Between-Group p-value | 0.05 |

TABLE 10
FEV₁ % Predicted (Day 0-Day 84)

| | Daclizumab | Placebo |
|---|---|---|
| | n = 88 (77%) | n = 27 (23%) |
| Baseline | | |
| Mean (s.e.) | 68.78 (1.2) | 68.00 (2.14) |
| N | 88 | 27 |
| Day 84 | | |
| Mean (s.e.) | 71.37 (1.38) | 67.57 (2.61) |
| N | 76 | 26 |
| Change from Baseline | | |
| Mean (s.e.) | 2.45 (0.98) | −0.97 (1.42) |
| N | 76 | 26 |
| Within-Group p-value | 0.02 | 0.50 |

| | |
|---|---|
| Between-Group p-value | 0.06 |

TABLE 11
FEV₁/ FVC (Day 0-Day 84)

| | Daclizumab | Placebo |
|---|---|---|
| | n = 88 (77%) | n = 27 (23%) |
| Baseline | | |
| Mean (s.e.) | 0.70 (0.01) | 0.71 (0.01) |
| N | 88 | 27 |
| Day 84 (L/sec) | | |
| Mean (s.e.) | 0.72 (0.01) | 0.70 (0.01) |
| N | 76 | 26 |
| % Change from Baseline | | |
| Mean (s.e.) | 0.02 (0.01) | −0.02 (0.01) |
| N | 76 | 26 |
| Within-Group p-value | 0.002 | 0.39 |

| | |
|---|---|
| Between-Group p-value | 0.01 |

TABLE 12
FEF (25-75%) (Day 0-Day 84)

| | Daclizumab | Placebo |
|---|---|---|
| | n = 88 (77%) | n = 27 (23%) |
| Baseline (L/sec) | | |
| Mean (s.e.) | 1.74 (0.09) | 1.64 (0.1) |
| N | 88 | 27 |
| Day 84 (L/sec) | | |
| Mean (s.e.) | 1.85 (0.1) | 1.55 (0.11) |
| N | 76 | 26 |
| Change from Baseline | | |
| Mean (s.e.) | 0.16 (0.05) | −0.10 (0.09) |
| N | 76 | 26 |
| Within-Group p-value | 0.002 | 0.63 |

| | |
|---|---|
| Between-Group p-value | 0.005 |

Each of the four different protocol-specified analyses of patient spirometry data listed in Tables 9-12 (FEV1, FEV1% predicted, FEV1/FVC, and FEF (25-75%) indicates a significant difference in favor of daclizumab of the mean change from baseline to Day 84 of the study.

Furthermore, it was observed that between Day 14 and Day 112 of the study, the measured values for percent of baseline FEV₁ for daclizumab treated patients remained consistently above (by approximately 2-5%) the corresponding values for placebo.

TABLE 13
Symptom score (Sx) (Day 0-Day 84)

| | Daclizumab | Placebo |
|---|---|---|
| [0-24] | n = 88 (77%) | n = 27 (23%) |
| Baseline | | |
| Mean (s.e.) | 8.0 (0.5) | 9.7 (0.9) |
| N | 77 | 23 |
| Day 84 | | |
| Mean (s.e.) | 6.8 (0.6) | 9.9 (1.2) |
| N | 67 | 21 |
| Change from Baseline | | |
| Mean (s.e.) | −1.2 (0.4) | 0.1 (0.4) |
| N | 62 | 20 |
| Within-Group p-value | 0.002 | 0.79 |

| | |
|---|---|
| Between-Group p-value | 0.018 |

TABLE 14
β₂-agonist MDI use (Day 0-Day 84)

| | Daclizumab | Placebo |
|---|---|---|
| [#puffs/day] | n = 88 (77%) | n = 27 (23%) |
| Baseline | | |
| Mean (s.e.) | 4.1 (0.4) | 4.5 (0.6) |
| N | 71 | 20 |
| Day 84 | | |
| Mean (s.e.) | 2.9 (0.4) | 5.0 (0.9) |
| N | 55 | 18 |
| Change from Baseline | | |
| Mean (s.e.) | −0.9 (0.4) | 0.5 (0.3) |
| N | 49 | 16 |
| Within-Group p-value | 0.03 | 0.15 |

| | |
|---|---|
| Between-Group p-value | 0.009 |

TABLE 15
AM PEFR (Day 0-Day 84)

| | Daclizumab | Placebo |
|---|---|---|
| [L/min] | n = 88 (77%) | n = 27 (23%) |
| Baseline | | |
| Mean (s.e.) | 367.6 (10.9) | 344.6 (16.8) |
| N | 77 | 23 |
| Day 84 | | |
| Mean (s.e.) | 389.3 (12.2) | 348.8 (16.2) |
| N | 67 | 21 |
| Change from Baseline | | |
| Mean (s.e.) | 12.9 (3.8) | −0.1 (9.5) |
| N | 62 | 20 |
| Within-Group p-value | 0.001 | 0.99 |

| | |
|---|---|
| Between-Group p-value | 0.217 |

TABLE 16
PM PEFR (Day 0-Day 84)

| | Daclizumab | Placebo |
|---|---|---|
| [L/min] | n = 88 (77%) | n = 27 (23%) |
| Baseline | | |
| Mean (s.e.) | 359.4 (10.6) | 329.6 (14.0) |
| N | 79 | 23 |
| Day 84 | | |
| Mean (s.e.) | 374.5 (12.2) | 326.7 (13.1) |
| N | 61 | 21 |
| Change from Baseline | | |
| Mean (s.e.) | 15.1 (4.1) | −4.1 (7.3) |
| N | 57 | 19 |
| Within-Group p-value | <0.001 | 0.77 |

| | |
|---|---|
| Between-Group p-value | 0.029 |

The protocol-specified analyses of patient data listed in Tables 13-16 also supported efficacy of daclizumab treatment. As shown by the results in Tables 13 and 14, during Treatment Period 1 there were significant reductions in symptoms (Sx) and β2-agonist rescue use for the daclizumab treated group. Also, as shown by the results in Table 16, the mean change from baseline in evening peak flow rate (PM_PEFR) over Treatment Period 1 significantly favored daclizumab treated patients relative to placebo treated patients. In addition, statistically significant results in favor of the daclizumab treated group were obtained for the continuous variable endpoint, %_FEV1.

Statistically significant data was not obtained for the continuous variables, AM_PEFR, and %Days Sx_Free, however, the mean change from baseline to Day 84 for both of these endpoints did favor the daclizumab treated patients relative to placebo. This general trend in favor of daclizumab further supports its efficacy for treatment of asthma.

TABLE 17
Exacerbations

| | Daclizumab | Placebo | |
|---|---|---|---|
| | n = 88 (77%) | n = 27 (23%) | p-value |
| Exacerbations | 6 of 76 (8%) | 4 of 26 (15%) | 0.37 |

As shown by the results in Table 17, measured exacerbation events were substantially lower (8% versus 15%) in the daclizumab treated patient group. Further, the Kaplan-Meier curve for time to exacerbation in daclizumab-treated patients was found to be significantly higher (in terms of survival distribution function) than the corresponding curve for placebo-treated patients. The increased survival distribution function values observed for daclizumab treated patients extended from times to exacerbation of approximately 14 days out to 225 days.

Similarly, the Kaplan-Meier curves for time to systemic corticosteroid use for daclizumab-treated patients was found to be significantly higher (in terms of survival distribution function) than the corresponding curve for placebo-treated patients. In this case, the increased survival distribution function values observed for daclizumab treated patients extended from times to exacerbation of approximately 42 days out to 250 days.

3. Pharmacodynamic Endpoint

Table 18 lists results obtained based on pharmacodynamic data and endpoints. In addition, it was observed that the percent of baseline peripheral eosinophil levels in daclizumab treated patients remained substantially lower than the levels in placebo treated patients at all time points (Days 28, 56 and 84) between Days 0 and 84.

TABLE 18
Peripheral Eosinophils (Day 0-Day 84)

| | Daclizumab | Placebo |
|---|---|---|
| [k/mm3] | n = 88 (77%) | n = 27 (23%) |
| Baseline | | |
| Mean (s.e.) | 0.2 (0.02) | 0.22 (0.02) |
| N | 87 | 26 |
| Day 84 | | |
| Mean (s.e.) | 0.14 (0.01) | 0.23 (0.03) |
| N | 74 | 25 |
| Change from Baseline | | |
| Mean (s.e.) | −0.05 (0.02) | 0.02 (0.03) |
| N | 73 | 24 |
| Within-Group p-value | 0.003 | 0.45 |

| | |
|---|---|
| Between-Group p-value | 0.04 |

4. Post-Hoc Analyses

Results were also obtained in terms of log odds ratio for the dichotomized variables: % exacerbations, decreased Sx>=25%, and increased FEV1>=10%. All three variables exhibited log odds ratios of approximately 1 in favor of daclizumab treated patients. Although the data for each was not statistically significant, this general trend in favor of daclizumab further supports its efficacy for treatment of asthma.

5. Pharmacokinetics (PK)

The main PK parameters determined in the Phase II study are listed in Table 19.

TABLE 19
Summary of main PK parameters

| | V1 | CL | Cₘₐₓ | Beta_HL | C_{ssavg} | AUC_{0-inf} | Vₛₛ |
|---|---|---|---|---|---|---|---|
| | (mL/kg) | (mL/hr/kg) | (μg/mL) | (hr) | (μg/mL) | (hr * μg/mL) | (mL/kg) |
| Median | 48.6 | 0.1297 | 39.9 | 432 | 22.9 | 82892 | 77.9 |
| Mean | 47.0 | 0.1339 | 42.2 | 473 | 23.9 | 86117 | 79.5 |
| SD | 12.7 | 0.0448 | 12.6 | 157 | 7.4 | 29493 | 25.5 |
| CV % | 27.0% | 33.5% | 29.9% | 33.3% | 31.2% | 34.2% | 32.1% |
| N* | 16 | 32 | 16 | 32 | 32 | 32 | 16 |

Thirty-two patients were evaluated for PK modeling. The results indicate low clearance, low volume of distribution, and a long elimination half-life of approximately 20 days. The initial low volume of distribution close to plasma volume indicates no initial drug distribution outside circulation. No drug accumulation was observed after the loading dose.

One clinical site (involving 6 patients) sampled the blood from the same location where drug was administered. This resulted in a biased high concentration for 5 minute post dose sample values and has a significant impact on the calculation of Cₘₐₓ, V1 and Vₛs values. All of the 6 patients from this site were excluded from the statistics for V1, Vₛₛ and first dose Cₘₐₓ values.

A modeled curve for the group mean (i.e. a semi-log scale plot of the simulated group mean PK profile) showed a very close correlation with the observed mean PK values. After the final dosing at day 126, the observed PK values decreased to approximately 1.5 μg/mL serum concentration of daclizumab by day 210. This decrease was in close correlation with the modeled curve which decrease down to 1 μg/mL serum concentration at approximately day 214.

6. Immunogenicity

Serum samples screened for anti-daclizumab antibodies using bridging ELISA. Positive samples from screening were then further evaluated in confirmatory assay. Of 113 patients tested for anti-daclizumab antibodies, 10 patients screened positive in the screening assay. Of these, 6 were confirmed positive in the daclizumab-specific confirmatory assay. Of those patients receiving daclizumab, 4.7% (4/86) were confirmed for detectable antibodies.

7. Conclusions

The above-described results of the Phase II study demonstrate that daclizumab is efficacious in treating adult patients with chronic, persistent asthma who are suboptimally controlled on inhaled corticosteroids. Specifically, patients showed improved pulmonary function (spirometry) as assessed by the percent change from baseline in FEV₁ during Treatment Period 1 (Day 0 to Day 84). Other spirometric measures were consistent with these results. Statistically significant within-group and between-group changes from baseline were revealed in diary symptom scores, β2-agonist rescue use, and nighttime peak expiratory flow rates. There were no significant within-group changes seen in the placebo group.

Also, patients receiving daclizumab demonstrated a statistically significant increase in the time to asthma exacerbation requiring oral corticosteroid rescue. Furthermore, peripheral eosinophil counts were significantly reduced in the daclizumab treated group, and there was a clear and consistent signal across all clinical pharmacodynamic endpoints.

In addition, treatment with daclizumab was generally well tolerated. The overall frequency and severity of adverse events did not differ between daclizumab and placebo groups.

### Example 2

### Results of Phase II Study Data Including Inhaled Steroid Taper During Treatment Period 2.

This example describes the efficacy results based on the data obtained out to Day 140 for the Phase II study described in Example 1 (Protein Design Lab Protocol No. DAC-1003). Briefly, starting at Day 85 (i.e. beginning of Treatment Period 2), patients had their inhaled triamcinolone (TAA) dose reduced by 25% of the dose on which they completed Treatment Period 1 at two-week intervals (Days 84, 98, and 112) until they had completely eliminated inhaled steroids (Day 126) while receiving infusions of daclizumab 1 mg/kg or placebo (Days 84, 98, 112, and 126), infused over a 15-minute period. As described in Example 1 (see also FIG. 3 for summary), patients were seen every two weeks for dosing, assessments, and efficacy measurements.

Results

Patients on daclizumab demonstrated prolonged time to exacerbation requiring systemic steroid rescue compared to placebo group patients (p=0.024). Furthermore, exacerbation rates were reduced in the daclizumab group compared to the placebo group for the 20-week steroid-stable and steroid-taper phases (11.6% vs. 28.6%, p=0.09). Patients on daclizumab demonstrated decreased peripheral eosinophil counts from baseline to week 20 (−30±20 /mm³ vs. placebo +60±30/mM³, p=0.004). During the first 56 days, daclizumab-treated patients with elevated baseline serum eosinophil cationic protein (sECP) had a significant reduction in sECP from baseline compared to placebo (p<0.01). Peripheral eosinophils decreased significantly in daclizumab patients who had no asthma exacerbations compared to an increase among daclizumab-treated patients with at least one exacerbation (p=0.032).

### Example 3

### Analysis of Severe Asthma Patient Subset

The Phase II study of daclizumab efficacy for treatment of asthma described in Example 1 was analyzed specifically for efficacy in a subset of patients with severe asthma. The patient whose data was used for this analysis were those with “refractory asthma” as defined in “Proceedings of the ATS workshop on refractory asthma: current understanding, recommendations, and unanswered questions,” American Thoracic Society,” Am J Respir Crit Care Med. 162(6):2341-51 (2000). The defined parameters are: TAA dose >200 mcg/day, asthma symptoms requiring short acting β-agonist use on a daily or near daily basis and FEV1<80% predicted. Thirty-three patients from the Phase II study met this definition of refractory asthma. The week 12 efficacy results for this refractory asthma subset are presented in Table 20 below. These results support the conclusion that daclizumab exhibits even greater efficacy (versus placebo) in severe asthma patients, as compared to the overall asthma patient population for which results are presented in Example 1, above.

TABLE 20
Efficacy analysis in severe asthma patient population

| | Daclizumab | | Placebo | | |
|---|---|---|---|---|---|
| | (N = 27) | | (N = 6) | | |
| Efficacy Measure | | N | | N | p-value |
| FEV1 | +8.5% | 21 | −9.3% | 6 | .01 |
| Asthma free days | 15% | 26 | 0% | 6 | .015 |
| PM_PEFR (L/min) | +15.7 | 18 | −13.5 | 6 | .028 |
| Symptoms (Sx) | −0.5 | 19 | +1.2 | 6 | .1 |
| β2-agonist MDI use | +0.3 | 17 | +1.3 | 6 | .404 |
| (puff/day) | | | | | |
| AM_PEFR (L/min) | +9 | 19 | −10 | 6 | .138 |

Asthma exacerbation data was also analyzed for the severe asthma patient population. The results for Treatment Period 1 (Day 0-Day 84), presented in Table 21, indicate a lower occurrence rate for exacerbations in the daclizumab-treated severe asthma patients (versus placebo).

TABLE 21
Asthma exacerbation in the severe asthma population

| | Daclizumab | Placebo | |
|---|---|---|---|
| | (N = 27) | (N = 6) | p-value* |
| Patients Reporting | 3/21 (14.3%) | 2/6 (33.3%) | 0.303 |
| Exacerbation | | | |
| Patients Reporting | 2/20 (10.0%) | 2/6 (33.3%) | 0.218 |
| Exacerbation Requiring | | | |
| CCS rescue | | | |

*p-value is based on Fisher's exact test.

Although the invention has been described with reference to the presently preferred embodiments, it should be understood that various modifications may be made without departing from the spirit of the invention. All publications, patents, patent applications, and web sites are herein incorporated by reference in their entirety to the same extent as if each individual patent, patent application, or web site was specifically and individually indicated to be incorporated by reference in its entirety.

1. A method of treating a respiratory disease in a patient in need of such treatment, comprising administering to said patient a therapeutically effective amount of a pharmaceutical formulation comprising an antibody that binds specifically to an IL-2 receptor.

2. The method of claim 1, wherein the respiratory disease is selected from the group consisting of asthma, allergic rhinitis, atopic dermatitis, nasal polyposis, Churg-Strauss syndrome, sinusitis, and COPD.

3. The method of claim 1, wherein said antibody is a humanized antibody.

4. The method according to claim 3, wherein said humanized antibody is daclizumab.

5. The method according to claim 1, wherein said antibody binds to the same epitope as daclizumab.

6. The method according to claim 5, wherein said antibody has an amino acid sequence that is at least 80% identical to the amino acid sequence of daclizumab.

7. The method according to claim 1, wherein the pharmaceutical formulation is administered parenterally, intravenously, intramuscularly, or subcutaneously.

8. The method of claim 7, wherein the pharmaceutical formulation is a liquid comprising about 100 mg/ml daclizumab, about 20-60 mM succinate buffer having pH from about 5.5 to about 6.5, about 0.01% -0.1% polysorbate, and a tonicity buffer that contributes to isotonicity.

9. The method according to claim 1, wherein said therapeutically effective amount is between about 0.001 mg/kg to 10 mg/kg.

10. The method according to claim 1, wherein said therapeutically effective amount is between about 0.5 mg/kg to 4.0 mg/kg.

11. The method according to claim 1, wherein said therapeutically effective amount is a fixed dose of between about 100 mg and 200 mg.

12. A method of treating asthma in a patient comprising: administering to said patient a therapeutically effective amount of a pharmaceutical formulation comprising an antibody that binds specifically to an IL-2 receptor.

13. The method according to claim 12, wherein said asthma is chronic, persistent asthma.

14. The method according to claim 12, wherein said asthma is moderate to severe asthma.

15. The method according to claim 12, further comprising administering to the patient one or more agents selected from the group consisting of beclomethasone, budesonide, flunisolide, fluticasone, triamcinolone, mometasone and acetonide.

16. The method according to claim 12, wherein said antibody has a binding affinity for said human IL-2 receptor of at least 10⁸ M⁻¹.

17. The method according to claim 12, wherein said antibody has a binding affinity for said human IL-2 receptor of at least 10⁹ M⁻¹.

18. The method according to claim 12, wherein said antibody is a monoclonal antibody.

19. The method according to claim 12, wherein said antibody is a chimeric antibody.

20. The method according to claim 12, wherein said antibody is a human antibody.

21. The method according to claim 12, wherein said antibody is a humanized antibody.

22. The method according to claim 21, wherein said humanized antibody is daclizumab.

23. The method according to claim 12, wherein said antibody binds to the same epitope as daclizumab.

24. The method according to claim 23, wherein said antibody has an amino acid sequence that is at least 80% identical to the amino acid sequence of daclizumab.

25. The method according to claim 24, wherein said antibody has CDR regions that have amino acid sequences that are at least 95% identical to the amino acid sequences of the CDR regions of daclizumab.

26. The method according to claim 12, wherein the pharmaceutical formulation is administered parenterally, intravenously, intramuscularly, or subcutaneously.

27. The method of claim 22, wherein the pharmaceutical formulation is a liquid comprising about 100 mg/ml daclizumab, about 20-60 mM succinate buffer having pH from about 5.5 to about 6.5, about 0.01% -0.1% polysorbate, and a tonicity buffer that contributes to isotonicity.

28. The method according to claim 12, wherein said therapeutically effective amount is between about 0.001 mg/kg to 10 mg/kg.

29. The method according to claim 12, wherein said therapeutically effective amount is between about 0.5 mg/kg to 4.0 mg/kg.

30. The method according to claim 12, wherein said therapeutically effective amount is a fixed dose of between about 100 mg and 200 mg.

31. A method of treating a Th2-cell mediated allergic disease in a patient in need of such treatment, comprising administering to said patient a therapeutically effective amount of a pharmaceutical formulation comprising an antibody that binds specifically to an IL-2 receptor.

32. The method of claim 31, wherein the disease is selected from the group consisting of asthma, allergic rhinitis, atopic dermatitis, nasal polyposis, Churg-Strauss syndrome, and sinusitis.

33. The method of claim 31, wherein said antibody is a humanized antibody.

34. The method according to claim 33, wherein said humanized antibody is daclizumab.

35. The method according to claim 33, wherein said antibody binds to the same epitope as daclizumab.

36. The method according to claim 33, wherein said antibody has an amino acid sequence that is at least 80% identical to the amino acid sequence of daclizumab.
